(19) United States
(12) Patent Application Publication
(10) Pub. No.: US 2023/0000904 A1
(43) Pub. Date: Jan. 5, 2023

(54) ANTI-HEMORRHAGING COMPOSITIONS

(71) Applicant: Ariel Scientific Innovations Ltd., Ariel (IL)

(72) Inventors: Danny BARANES, Yad Hana (IL); Orly Eva WEISS, Petach-Tikva (IL); Roni HENDLER, Jerusalem (IL)

(73) Assignee: Ariel Scientific Innovations Ltd., Ariel (IL)

(21) Appl. No.: 17/902,949

(22) Filed: Sep. 5, 2022

Related U.S. Application Data
(63) Continuation of application No. PCT/IL2021/050244, filed on Mar. 5, 2021.
(60) Provisional application No. 62/985,345, filed on Mar. 5, 2020.

(51) Int. Cl. A61K 33/10; A61P 7/04; A61K 9/14; A61K 47/64; A61K 38/39

(52) U.S. Cl. CPC A61K 33/10; A61P 7/04; A61K 9/14; A61K 47/645; A61K 38/39

(57) ABSTRACT

Composite materials made of a citrate, a calcium carbonate-containing material and an association moiety which is associated with the citrate and the calcium carbonate-containing material are provided. The composite materials are typically particulate materials (e.g., powdery materials). Compositions and articles-of-manufacturing containing and/or configured for applying the composite materials are also provided, as well as their use in inducing blood coagulation and arresting hemorrhage, including internal and/or massive hemorrhage.

### RELATED APPLICATIONS

This application is a Continuation of PCT Patent Application No. PCT/IL2021/050244 having International filing date of Mar. 5, 2021, which claims the benefit of priority under 35 USC § 119(e) of U.S. Provisional Patent Application No. 62/985,345 filed on Mar. 5, 2020. The contents of the above applications are all incorporated by reference as if fully set forth herein in their entirety.

### FIELD AND BACKGROUND OF THE INVENTION

The present invention, in some embodiments thereof, relates to medicine and, more particularly, but not exclusively, to novel compositions which are useful in inducing blood coagulation and/or in reducing or blocking hemorrhaging, for example, arterial and internal massive hemorrhaging.

Hemorrhaging, or bleeding, is a term used to describe a condition in which blood escapes from the circulatory system. Bleeding can occur internally, where blood leaks from blood vessels inside the body, or externally, either through a natural opening such as the mouth, nose, ear, urethra, vagina or anus, or through a break in the skin. By “internal hemorrhaging” it is meant that a blood vessel inside the body is injured and leaks. Blood leakage from internal blood vessels can be manifested also as external hemorrhaging, yet the injured blood vessel, which is the source of hemorrhaging, is internal.

Coagulation, also known as clotting, is the process by which liquid blood forms a clot. Coagulation may result in hemostasis, a term known to describe the cessation of blood loss from a damaged vessel. The mechanism of coagulation, as is well-described in the art, involves both activation, adhesion, and aggregation of platelets and deposition and maturation of fibrin.

An anti-hemorrhagic agent is a substance that promotes hemostasis and arrests bleeding, and is also referred to in the art as a hemostatic agent, a hemostat, or as a pro-coagulant.

Internal hemorrhaging (or bleeding) due to blunt or penetrating, civilian or military, trauma is known to cause major loss of human life, as well as drainage of hospital and blood bank resources. Among the most lethal injuries are civilian and military injuries that cause internal hemorrhaging.

Common traumas that lead to internal hemorrhaging include injuries of solid abdominal organs such as liver, spleen, kidneys and other organs. These injuries are commonly treated by surgical techniques such as suturing, resection and devascularization of the organ. A large number of liver, spleen and renal injuries do not respond to these techniques, leading to death of the patient or sacrifice of a valuable organ.

Moreover, sever internal hemorrhaging can lead to rapid and heavy loss of blood and death prior to the availability of the surgical treatment. According to the U.S. Army Institute of Surgical Research (www(dot)defensemedianetwork(dot)com/stories/wound-care-and-healing), most death cases among soldiers are due to blood loss, or hemorrhage.

Bleeding injuries due to trauma or tissue damage during chirurgical operations may cause shock, contamination and death.

Currently used anti-hemorrhagic agents include systemic drugs, which act by inhibiting fibrinolysis or promoting coagulation. Administration of such medications is associated with the risk of generating embolism that may lead to stroke or death.

Anti-hemorrhagic, or hemostatic, agents are typically used during surgical procedures to achieve hemostasis.

Locally-acting hemostatic agents act by causing vasoconstriction or promoting platelet aggregation, and have been gaining popularity for use in emergency bleeding control, particularly for internal hemorrhaging caused by severe trauma, which is typically associated with massive bleeding.

Since systemic drugs are associated with a risk and since chirurgical procedures are often not applicable at the trauma site, blocking external and internal hemorrhaging by techniques which can be applied within a short time upon the injury is highly sought for, with the goal being agents or compositions which can arrest massive bleeding within several minutes from the injury and preserve the blockage for at least a few hours, for example, until a surgical treatment can be provided to the injured subject (e.g., until the subject is brought to a hospital).

Exemplary known hemostats include microfibrillar collagen hemostat (MCH), which is a topical agent composed of resorbable microfibrillar collagen, typically used in surgical procedures; Chitosan hemostats, which are also topical agents composed of chitosan and its salts, act by bonding with platelets and red blood cells to form a gel-like clot which seals a bleeding vessel, and are known to be used to stop traumatic life-threatening bleeding; zeolites, such as the product QuikClot Combat Gauze® (nonwoven gauze impregnated with kaolin , and Veriset, a gauze containing lysine, both act as absorbents, and are used for sealing severe injuries quickly; Thrombin and fibrin glue products are used surgically to treat bleeding and to thrombose aneurysms; desmopressin is used to improve platelet function by activating arginine vasopressin receptor 1A; Tranexamic acid and aminocaproic acid, present in products such as Hexakapron®, inhibit fibrinolysis, and lead to a de facto reduced bleeding rate. Some foam-forming agents have also been developed, which, once applied, form a foam that physically reduces bleeding by applying pressure to the blood vessels. The formed foam should thereafter be surgically removed.

Properties associated with the function of the exemplary commercially available products that act as locally-acting hemostatic agents QuikClot Combat Gauze® (kaolin), coagulation factor VII (F7), Veriset (Lysine) and Hexakapron® (Tranexamic acid) are summarized in Table A below. As can be seen, none of these products can arrest massive bleeding.

TABLE A

| | Combat Gauze | | Tranexamic acid | Veriset |
|---|---|---|---|---|
| Property | (Kaolin) | F7 | (Hexakapron) | (Lysine) |
| Stops massive | − | − | − | − |
| bleeding | | | | |
| Accelerates | + | + | − | − |
| coagulation | | | | |
| Blocks | − | − | + | + |
| Fibrinolysis | | | | |
| Topical | + | − | − | + |
| Non-exothermic | − | + | + | + |
| Tissue adhesive | ND | − | − | + |

Citrate salts are known anti-coagulation agents. Some compositions containing a citrate salt in combination with pH-adjusting agents such as sodium bicarbonate, or sodium carbonate, have been described, in which the sodium carbonate or bicarbonate are used to prolong the anti-coagulation activity of citrate by maintaining a non-acidic pH.

WO 2015/166497, by the present assignee, describes that calcium carbonate, for example, in the form of aragonite extracted from the skeleton of the corals, acts as an anti-coagulation or de-coagulation agent.

U.S. Pat. No. 5,985,315 describes a device for isolation of blood coagulation components using coral skeleton made of calcium carbonate. Some of the blood fractions passed through the device are described therein as human blood anti-coagulated by citrate.

WO 2017/046809, by the present assignee, describes blood coagulation-inducing compositions comprising a citrate salt and a calcium-carbonate containing particulate material.

Additional background art includes WO 2014/183886 and U.S. Patent Application Publication No. 2001/055621.

### SUMMARY OF THE INVENTION

According to an aspect of some embodiments of the present invention there is provided a composite material comprising a citrate, a calcium carbonate-containing material, and an associating moiety being associated with the citrate and the calcium carbonate-containing material.

According to some of any of the embodiments described herein, the associating moiety is a positively-charged moiety at physiological pH.

According to some of any of the embodiments described herein, the association moiety is a polymeric moiety.

According to some of any of the embodiments described herein, the association moiety has a molecular weight in a range of 10 to 100 kDa.

According to some of any of the embodiments described herein, the association moiety has a molecular weight of at least 300 kDa.

According to some of any of the embodiments described herein, the association moiety is a biocompatible moiety.

According to some of any of the embodiments described herein, the association moiety is a polypeptide.

According to some of any of the embodiments described herein, the polypeptide comprises at least one amino acid residue that is positively charged at physiological pH, or consists essentially of amino acids residues that are positively charged at physiological pH.

According to some of any of the embodiments described herein, the polypeptide is or comprises a polylysine.

According to some of any of the embodiments described herein, the polypeptide is or comprises a poly-D-lysine.

According to some of any of the embodiments described herein, the polylysine is selected poly-D-lysine, poly-L-lysine and poly-ε-lysine.

According to some of any of the embodiments described herein, the polypeptide is or comprises collagen.

According to some of any of the embodiments described herein, the association moiety is lysine (e.g., L-lysine and/or D-lysine and/or ε-Lysine).

According to some of any of the embodiments described herein, the association moiety is capable of affecting blood coagulation in a subject.

According to some of any of the embodiments described herein, the association moiety is capable of interfering in (e.g., inhibiting) a fibrinolysis process in a subject.

According to some of any of the embodiments described herein, the calcium carbonate-containing material comprises crystalline calcium carbonate.

According to some of any of the embodiments described herein, the calcium carbonate-containing material comprises a coral exoskeleton.

According to some of any of the embodiments described herein, the calcium carbonate-containing material comprises acellular coral exoskeleton.

According to some of any of the embodiments described herein, the calcium carbonate-containing material comprises aragonite.

According to some of any of the embodiments described herein, the calcium carbonate-containing material comprises biogenic aragonite.

According to some of any of the embodiments described herein, the calcium carbonate-containing material comprises geological aragonite.

According to some of any of the embodiments described herein, the calcium carbonate-containing material comprises amorphous calcium carbonate (ACC).

According to some of any of the embodiments described herein, the calcium carbonate-containing material is a particulate material.

According to some of any of the embodiments described herein, the particulate material comprises particles having an average particle diameter in the range of from 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns.

According to some of any of the embodiments described herein, the particulate material comprises particles having an average particle diameter in the range of from 0.1 micron to 100 microns, or from 0.1 microns to 50 microns.

According to some of any of the embodiments described herein, the particulate material comprises particles having an average diameter in the range of from 100 microns to 10 millimeter, or from 100 microns to 1 millimeter.

According to some of any of the embodiments described herein, at least a portion of the association moiety is deposited onto a surface of the particulate material.

According to some of any of the embodiments described herein, at least a portion of the citrate is associated with the portion of association moiety which is deposited onto a surface of the particulate material.

According to some of any of the embodiments described herein, at least a portion of the calcium carbonate-containing material is associated with at least a portion of the association moiety via electrostatic interactions formed between the carbonate of the calcium carbonate-containing material and a positively charged group of the association moiety.

According to some of any of the embodiments described herein, at least a portion of the citrate is associated with at least a portion of the association moiety via electrostatic interactions and/or hydrogen bond interactions.

According to some of any of the embodiments described herein, a weight ratio of the citrate and the calcium carbonate-containing material ranges from 10:1 to 1:10, or from 5:1 to 1:5.

According to some of any of the embodiments described herein, a weight ratio of the association moiety and the calcium carbonate-containing material ranges from 5000:1 to 250:1.

According to some of any of the embodiments described herein, the composite material further comprises a swelling polymeric moiety.

According to some of any of the embodiments described herein, the swelling polymeric moiety is selected from alginate, chitosan, collagen and a poly (alkylene glycol).

According to some of any of the embodiments described herein, the composite material further comprises a coagulating agent (a pro-coagulant such as fibrinogen and/or thrombin).

According to an aspect of some embodiments of the present invention there is provided a pharmaceutical composition comprising, or consisting of, the composite material as described herein in any of the respective embodiments and any combination thereof.

According to some of any of the embodiments described herein, the composition is formulated as a topical dosage form.

According to some of any of the embodiments described herein, the composition is in a form of a powder, a gel, a spray, a foam, a mousse, an ointment, a paste, a lotion, a gauze, a wound dressing, a suspension, an adhesive bandage, a non-adhesive bandage, a wipe, a gauze, a pad, and a sponge.

According to some of any of the embodiments described herein, the composition is in a form of a powder, preferably a dispersible powder.

According to an aspect of some embodiments of the present invention there is provided an article-of-manufacturing comprising the composite material or the composition of any of the respective embodiments and any combination thereof, the article-of-manufacturing being configured for applying the composite material or the composition to a bleeding organ and/or tissue.

According to some of any of the embodiments described herein, the article-of-manufacturing comprises a container for housing the composite material or the composite, and means for dispensing the composite material or the composition from the container onto a bleeding organ and/or tissue.

According to some of any of the embodiments described herein, the article-of-manufacturing comprises a substrate and the composite material or composition deposited in and/or on the substrate.

According to some of any of the embodiments described herein, the article-of-manufacturing is or comprises a bandage having the composite material deposited in and/or on a gauze.

According to an aspect of some embodiments of the present invention there is provided a composite material or a composition or an article-of-manufacturing as described herein in any of the respective embodiments and any combination thereof, for use in inducing coagulation of blood.

According to some of any of the embodiments described herein, the inducing coagulation of blood comprises contacting blood or a bleeding organ and/or tissue with the composition.

According to some of any of the embodiments described herein, at least 50% of the blood is clotted upon contacting with the composition for less than 10 minutes.

According to some of any of the embodiments described herein, at least 50% of the clotted blood remains clotted for at least 2 hours.

According to some of any of the embodiments described herein, the inducing coagulation of blood comprises contacting blood and/or the bleeding organ and/or tissue with the composite material or composition, the contacting being effected in vivo.

According to some of any of the embodiments described herein, the contacting further comprises applying pressure to the bleeding organ and/o tissue when contacted with the composite material or composition.

According to some of any of the embodiments described herein, applying the pressure is for a time period that ranges from 0.1 to 10 minutes.

According to some of any of the embodiments described herein, the contacting is with a blood vessel.

According to some of any of the embodiments described herein, the blood vessel is an internal blood vessel.

According to some of any of the embodiments described herein, the blood vessel is of an internal tissue.

According to some of any of the embodiments described herein, the tissue is selected from a hepatic tissue, a renal tissue, an abdominal tissue, a pancreatic tissue, a gastrointestinal tissue, a pulmonary tissue, a gonadal tissue, a spleen tissue, a skin tissue, a vascular tissue, and a nervous tis sue.

According to some of any of the embodiments described herein, inducing coagulation of blood is for reducing or arresting hemorrhaging in a subject in need thereof.

According to an aspect of some embodiments of the present invention there is provided a composite material or a composition or an article-of-manufacturing of any of the respective embodiments and any combination thereof, for use in reducing or arresting hemorrhaging in a subject in need thereof.

According to some of any of the embodiments described herein, the hemorrhaging is an internal hemorrhaging.

According to some of any of the embodiments described herein, the hemorrhaging is a massive hemorrhaging.

According to an aspect of some embodiments of the present invention there is provided a composite material or a composition or an article-of-manufacturing of any of the respective embodiments and any combination thereof, for use in treating traumatic brain injury.

According to some of any of the embodiments described herein, treating the traumatic brain injury comprises contacting the injury site with the composite material.

According to some of any of the embodiments described herein, the contacting comprises implanting the composite material in the injury site.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents possible pathways involved in blood coagulation that are affected by a composite material of the present embodiments.

FIG. 2A is a bar graph showing time quantification of the bleeding arrest upon application of various composite materials according to some of the present embodiments. C=citrate; ACC=amorphous calcium carbonate; CS=coral skeleton, PDL=poly-D-lysine.

FIG. 2B presents photographs (upper panel) and a bar graph (lower panel) showing the decrease in time to bleeding cessation (TBC) following ACC and CPC application to blood samples drawn from mice.

FIG. 3A presents photographs of a brain wound induced in a mouse, following (from left to right) wound induction, consequent bleeding, following deposition of an exemplary composite material according to some of the present embodiments (CPC), and subsequent removal of excess thereof.

FIG. 3B presents photographs of an abdominal injury induced in mice, before and following deposition of an exemplary composite material according to some of the present embodiments (denoted as CPC).

FIG. 4 presents photographs of an abdominal injury treated with a powder of an exemplary composite material according to some of the present embodiments (denoted as CPC), and washed with saline to remove excess powder.

FIGS. 5A-F are photographs of a swine femoral vein, marked by an arrow (FIG. 5A), of an injury thereof generated through a puncture by cannula (FIG. 5B), of the resulting bleeding, marked by an arrow (FIG. 5C), of the bleeding arrest upon application of an exemplary composite material according to the present embodiments (denoted as ACC-PDL-C) upon 2 minutes pressure application using a gauze (FIG. 5D), of the injured area 3 hours post application of ACC-PDL-C (FIG. 5E), and of the closed wound after removal of the ACC-PDL-C, 3 hours post-application, as marked by an arrow (FIG. 5F).

FIGS. 6A-C are photographs of the blocked injury site shown in FIGS. 5A-F, upon femur bending as marked by the arrow (FIG. 6A), upon leg bending (FIG. 6B) and after shaking the site (FIG. 6C), showing the resistance of the blocked injury to body shaking.

FIGS. 7A-D are photographs of the bleeding of an injured swine femoral vein (FIGS. 7A and 7C), of the bleeding arrest upon application of an ACC-PDL-C composite material 3 weeks (FIG. 7B) and 4 weeks (FIG. 7C) upon its preparation, followed by 4 minutes pressure application.

FIGS. 8A-B are photographs of the bleeding of an injured swine femoral vein (FIG. 8A), and upon application of a B-ACC-PDL-C composite material, followed by 4 minutes pressure application.

FIGS. 9A-C are photographs of a swine femoral vein, marked by an arrow (FIG. 9A), of the bleeding upon inducing injury in the swine femoral vein (FIG. 9B), and upon application of ACC-L-Lysine-C composite material followed by 2 minutes pressure application (FIG. 9C).

FIGS. 10A-B are photographs of the bleeding of an injured swine femoral vein (FIG. 10A), and upon application of a CS(large)-PDL-C, followed by 1 minute pressure application (FIG. 10B).

FIGS. 11A-D are photographs of the bleeding of an injured swine femoral artery (FIG. 11A), upon application of ACC-PDL-C composite material, followed by 4 minutes pressure application (FIG. 11B), of body bending (FIG. 11C), and of the blocked arterial injury after the bending (FIG. 11D).

FIGS. 12A-B are photographs of the bleeding of an injured swine liver (FIG. 12A), and upon application of ACC-PDL-C composite material, followed by 2 minutes pressure application (FIG. 12B).

FIGS. 13A-F are photographs of the bleeding of swine liver injuries being 3.5 cm in length, 0.4-0.5nun in depth (FIGS. 13A, 13C and 13E), and upon application of ACC-PDL-C composite material (FIG. 13B) to the injury shown in FIG. 13A, of B-ACC-PDL-C composite material (FIG. 13D) to the injury shown in FIG. 13C, and of a CS(small)-PDL-C composite material (FIG. 13F) to the injury shown in FIG. 13E).

FIGS. 14A-F are photographs of the bleeding of injured spleen of a swine (FIGS. 14A, 14C and 14E), and upon application of ACC-PDL-C composite material (FIG. 14B) to the injury shown in FIG. 14A, of CS(large)-PDL-C composite material (FIG. 14D) to the injury shown in FIG. 14C, and of a CS(small)-PDL-C composite material (FIG. 14F) to the injury shown in FIG. 14E).

FIGS. 15A-B are photographs of the bleeding of a superficial wound in a swine (FIG. 15A) and upon application of ACC-PDL-C followed by 2 minutes pressure application.

FIGS. 16A-B are photographs of a femoral vein injury site, marked by arrows, covered with ACC-L-Lysine-C (FIG. 16A) and of ACC-L-Lysine (with no citrate) (FIG. 16B).

FIG. 17 is a bar graph showing the effect of ACC, ACC+citrate, ACC+Poly-L-lysine (Polymer 2; MW—30-70 kDa), and CPC E on the time to bleeding cessation (TBC) of a 2.75 mm diameter injury in a swine's femoral vein.

FIGS. 18A-B presents a plot showing the time to bleeding cessation (TBC) of a 0.8 mm diameter injury in a swine's femoral vein upon application of various doses of the tested CPC (2.5, 5, 10, 15, 20, 25 grams), n=2 (FIG. 18A), and a bar graph showing the minimal amount of CPC required to achieve the shortest TBC as a function of wound injury (FIG. 18B).

FIGS. 19A-B are bar graphs showing the effect of grains size on the TBC of a liver injury is swine (n=2).

FIG. 19A shows the TBC upon application of CPC C, which has grain size higher than 40 micrometer (A) and of CPC C which has gran size lower than 40 micrometers (B); and FIG. 19B shows the effect of CPC D (B) versus that of ACC and citrate.

FIG. 20 is a bar graph showing the effect of composite materials CPC D (A), CPC E (B) and CPC F (C), having various associating moieties of different lengths (MW), on the bleeding cessation 15 minutes after induction of a 2.75 mm diameter injury in swine femoral vein.

FIG. 21 is a bar graph showing the effect of composite materials CPC E (A), CPC G (B), CPC H (C) and CPC I (D), having various types pf polymeric associating moieties, on a 2.75 mm diameter injury in swine femoral vein (n=1). Data is shown as TBC normalized to bleeding severity.

FIG. 22 is a bar graph showing the effect of 20 grams of composite materials Polymer D (CPC E) and Polymer L (CPC A), on the TBC of a 2.75 mm diameter injury in swine femoral vein (n=2; mean+SD).

FIG. 23 is a bar graph showing the effect of a CPC E-containing bandage on the TBC of a 2.75 mm diameter injury in swine femoral vein (n=1).

FIGS. 24A-C present photographs arranged to show the safety studies conducted in swine (FIG. 24A), and photographs obtained following histological measurement of a swine's vein following treatment (FIGS. 24 B-C, each for a different sample). In FIG. 24B, T is mural thrombus in the vein lumen; the asterisk (*) indicate the areas of powder accumulation in the adventitia, with neutrophil administration. Fibrin strands are also observed in the area. Magnification ×40. Scale bar: 500 microns. In FIG. 24C, the arrows indicate fibrin deposition in the intima, which indicate activation of the clotting cascade. Powder is noted in the adventitia. Inflammation is negligible. A focal aggregation of neutrophils is observed in the perivascular tissue, where there is edema and fibrin deposition. Magnification ×40. Scale bar: 500 microns.

FIGS. 25A-D present photographs showing a 3-month old mice brain following traumatic brain injury (FIG. 25A), an exemplary composite material according to some of the present embodiments (CP) implanted at the injury site (FIG. 25B), and bar graphs showing the effect of the implant on mice brain functions (FIGS. 25C-D).

FIGS. 26A-B present SV2 images of mice brain, following injury, and following treatment with an exemplary composite material according to some of the present embodiments (FIG. 26A), and a bar graph showing the average fluorescent intensity of the SV2 staining under the above conditions.

FIGS. 27A-B demonstrate the capacity of an exemplary composite material according to some of the present embodiments to cause platelet aggregation. FIG. 27A presents images of grains layers on glass coverslips exposed to blood for 3 minutes followed by Giemza staining; Arrow in the right panel points on stained platelets. FIG. 27B presents the same, upon immunolabeling with an antibody to the protein CD41, a platelet marker (red=platelets; green=grains).

FIGS. 28A-B show the effect of an exemplary composite material according to some of the present embodiments on platelet aggregation having collagen associated therewith. FIG. 28A are photographs showing that addition of the collagen changes the physical property of the composite from a disperse grains powder to a powder of more tightly associated grains. FIG. 28B shows immunofluorescence staining of the CPC-collagen associated platelets upon immunolabeling with an antibody to the protein CD41, showing a stronger effect of platelet aggregation compared to the composite alone shown in FIG. 27B.

### DESCRIPTION OF SPECIFIC EMBODIMENTS OF THE INVENTION

The present invention, in some embodiments thereof, relates to medicine and, more particularly, but not exclusively, to novel compositions which are useful in inducing blood coagulation and/or in reducing or blocking hemorrhaging, for example, venous, arterial and/or internal hemorrhaging.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As described in the Background section hereinabove, some of the present inventors have previously described, in WO 2017/046809, using a citrate salt and a calcium-carbonate containing particulate material such as a coral skeleton material, for inducing blood coagulation and arresting hemorrhage.

Without being bound by any particular theory, it has been assumed that when these two agents act together, the citrate promotes release of calcium ions from the calcium carbonate in an amount sufficient to stimulate coagulation. This effect is achieved by applying the two agents when mixed in a single composition or when administered individually either concomitantly or sequentially.

The present inventors have now conceived improving the coagulation effect induced by citrate salt and a calcium-carbonate containing particulate material, by physically combining these agents and have designed and successfully prepared and practiced a composite material (a conjugate) in which a citrate and a calcium-carbonate containing particulate material are linked to one another by means of an association moiety. As an exemplary association moiety, a polypeptide-based polymeric material, Poly-D-Lysine (PDL), and free lysine were first used, and then other, various associating moieties were also tested. Lysine has structural features which are similar to those of tranexamic acid, which acts as blood clot stabilizer by inhibition of plasmin and is used clinically to treat bleeding [Hunt B J. Anaesthesia. 2015 Jan;70 Suppl 1:50-3, e18].

As demonstrated in the Examples section that follows, the powdery composite material obtained by associating together particulate calcium carbonate, poly-D-lysine and citrate (CPC), when applied topically to a bleeding tissue or organ, induced coagulation in vitro and arrested massive abdominal and head bleedings in mice in a time scale of seconds to minutes. See, FIGS. 2A-4. The powdery composite material was shown to be hygroscopic and adhesive, and no exothermic reaction occurred upon its application.

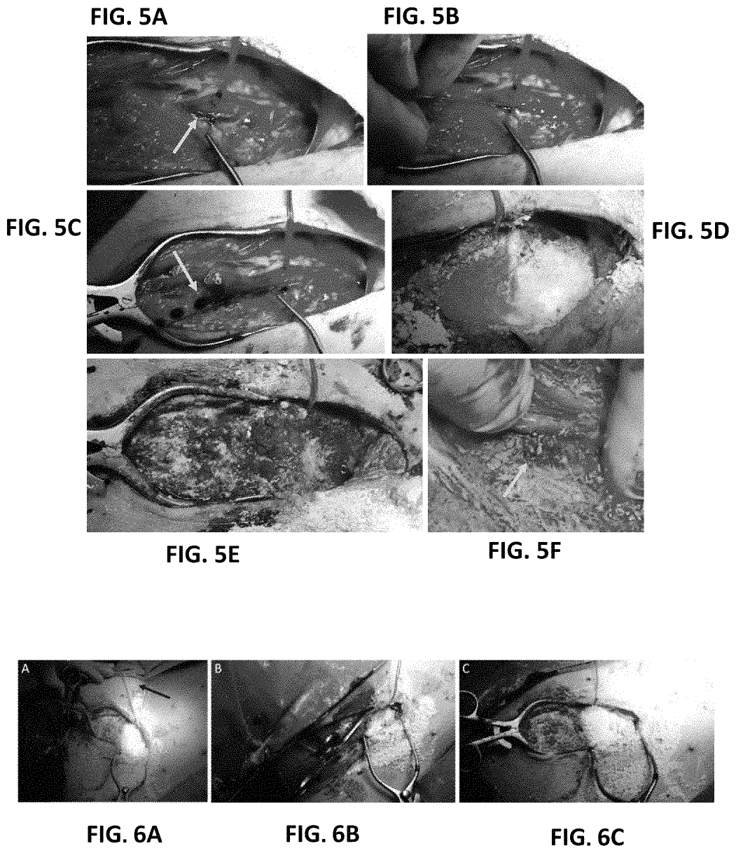

As further demonstrated in the Examples section that follows, the powdery composite material obtained by associating together particulate calcium carbonate and citrate with polymeric and nonpolymeric associating moieties, when applied topically to a bleeding tissue or organ (e.g., a blood vessel, liver or spleen), induced coagulation in vivo and arrested massive bleedings in swine, including of injured arteries, veins, liver and spleen, in a time scale of several seconds to minutes. See, FIGS. 5A-23 and Table 5.

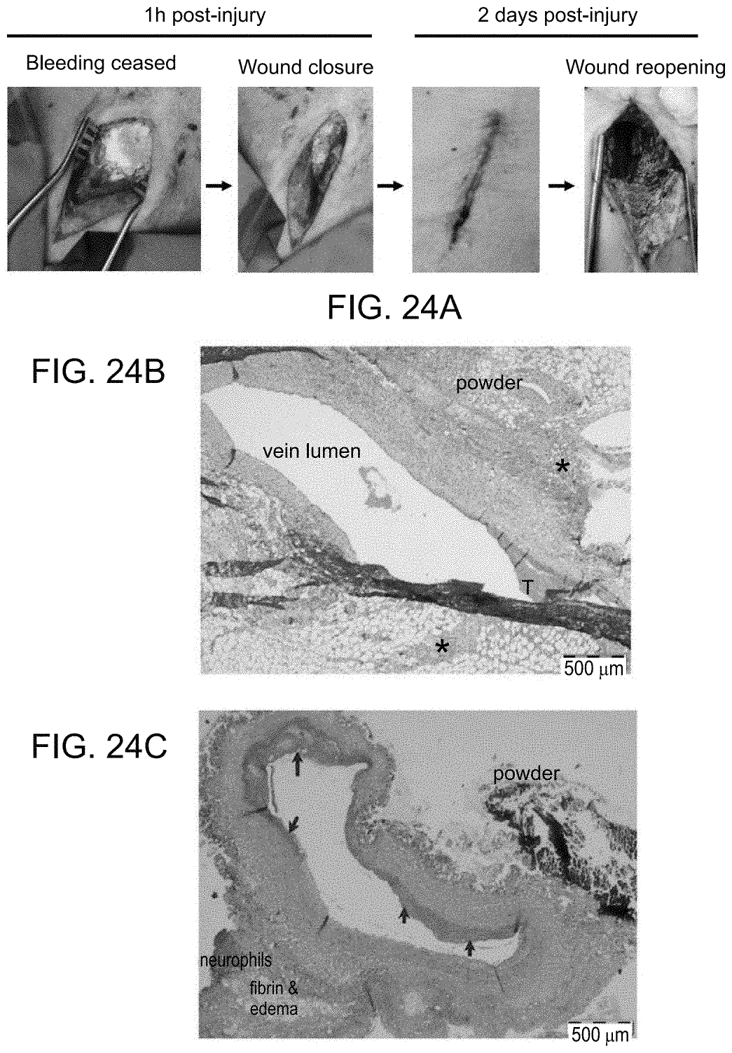

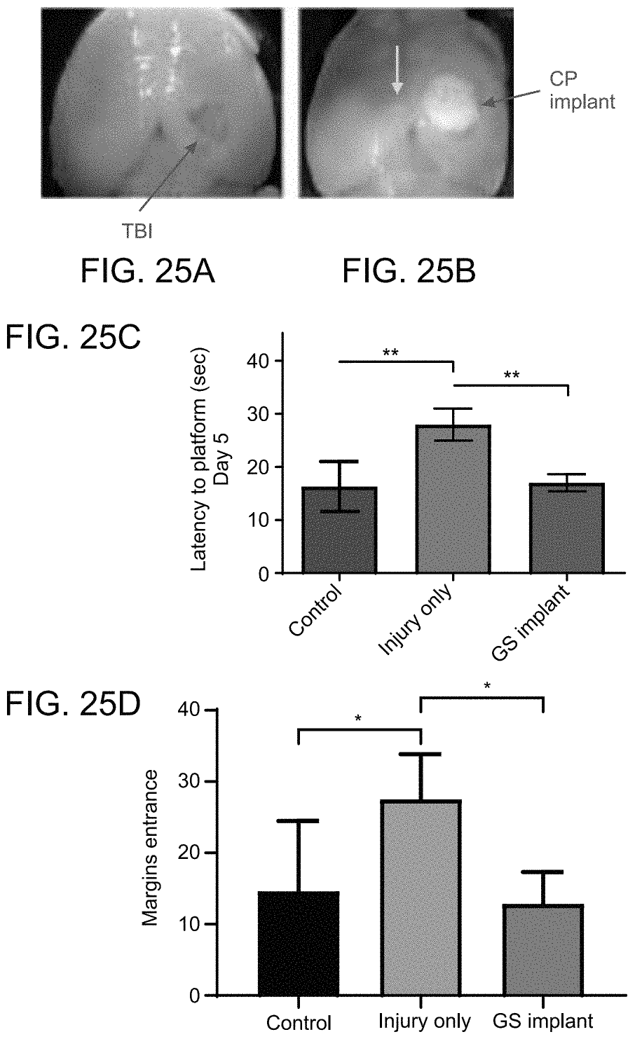

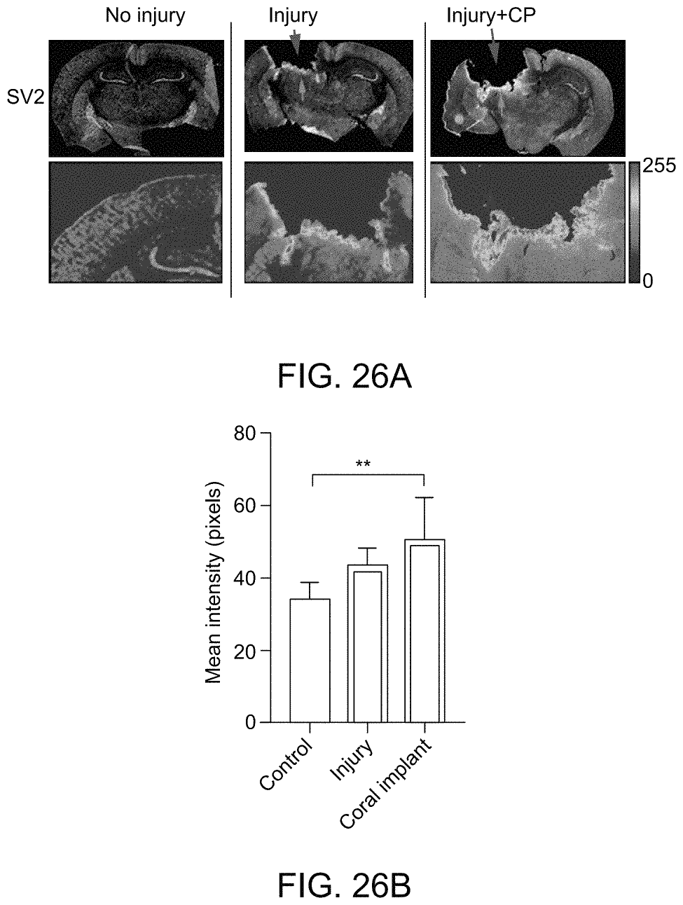

Further studies have showed that the composite material is non-toxic and safe for use (see, FIGS. 24A-C and Example 7), and, when applied to injured brain, the composite material is able to restore brain functions (see, FIGS. 25A-26B).

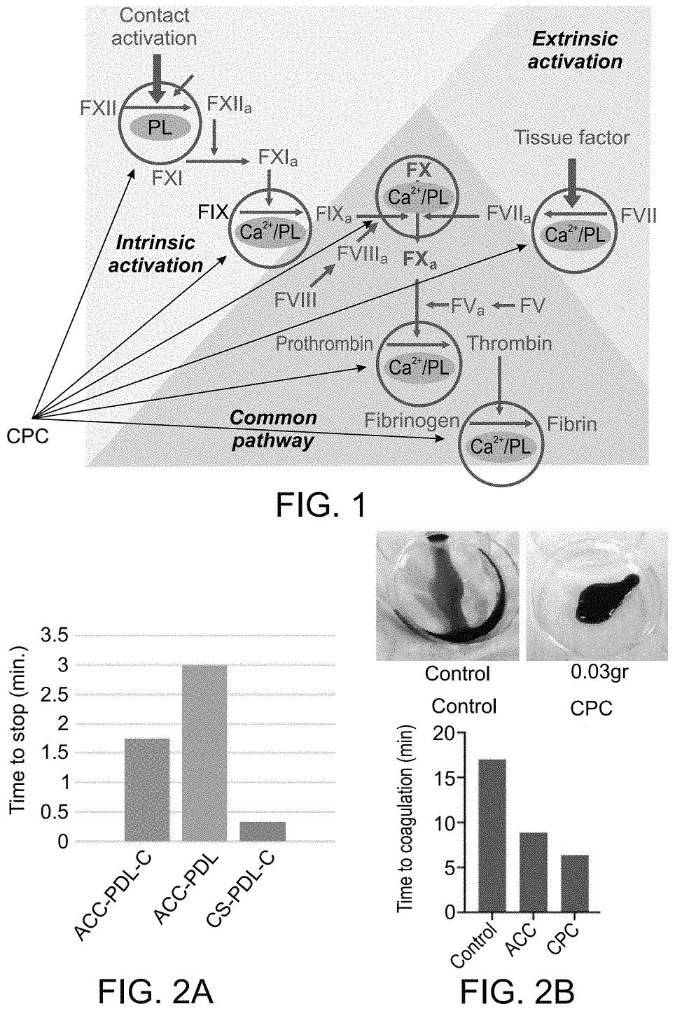

Without being bound by any particular theory, it is assumed that the strong effect of a composite material as described herein on blood coagulation arises from several different mechanisms and/or from their synergism, as shown in FIG. 1. One mechanism is associated with calcium activation of the coagulation cascade, that results from the possible citrate-induced release of calcium ions from the calcium carbonate-containing material. An additional mechanism may be associated with a selected association moiety, which may be such that causes stabilization of clots by reducing their degradation, for example, by inhibiting plasmin, the enzyme that reduces clots half-life by degrading fibrin. An additional mechanism may involve interactions with platelets, by the calcium carbonate and/or the association moiety, which promote the formation and stabilization of the blood clot. It is assumed that the latter, physical, non-biological mechanism is the primary act of the composite material, as it is fastest than the biological actions and hence the first to occur upon application, and as it accelerates blood coagulation by itself.

Overall, it is assumed that the herein disclosed composite material features all of the properties presented in Table A above, contrary to currently available products for treating hemorrhage, and can be utilized as a life-saving technology that enables rapid arrest of massive hemorrhage caused by severe injury or during operation, which cannot be blocked by non-chirurgical means.

Embodiments of the present invention therefore relate to novel composite materials, to compositions, articles-of-manufacturing and kits comprising same, and to uses thereof in inducing blood coagulation and in arresting hemorrhage, including massive hemorrhage and internal hemorrhage.

The Composite Material:

According to an aspect of some embodiments of the present invention there is provided a composite material which comprises a calcium carbonate-containing material, a citrate and an associating moiety being associated with the citrate and the calcium carbonate-containing material.

By “associated with” it is meant physical and/or chemical association between the indicated components.

Thus, for example, the citrate and/or association moiety can be attached to the calcium carbonate-containing material, by interacting with the carbonate groups and/or the calcium cations via, e.g., covalent bonds, electrostatic interactions, hydrogen bonding, van der Waals interactions, donor-acceptor interactions, and/or cation-7c interactions. These interactions lead to the chemical association between the components.

As an example, and without being bound by any particular theory, the citrate ions may be in chemical association with the positively charged calcium of the calcium carbonate, and/or with positively charged groups of the association moiety. The carbonate groups of the calcium carbonate can also be in chemical association with positively charged groups of the association moiety, while the calcium can be in chemical association with negatively charged group of the association moiety.

Alternatively, the components can be attached to one another by physical association such as surface adsorption, encapsulation, entrapment, entanglement and the likes.

The Calcium-Carbonate Containing Material:

Herein throughout, the phrase “calcium carbonate-containing material” describes a material, a substance or a composition-of-matter, which is substantially consisted of calcium carbonate, that it, which includes at least 50%, or at least 60%, or at least 70%, or at least 80%, or at least 90%, or at least 95%, or about 100%, by weight, calcium carbonate.

The term “calcium carbonate” as used herein, refers to the chemical compound CaCO₃. In some embodiments, the calcium carbonate is solid calcium carbonate, which can be in crystalline or amorphous form. As used herein, crystalline forms of calcium carbonate include aragonite, calcite, ikaite, vaterite and monohydrocalcite, all of which are encompassed. Other solid forms of calcium carbonate include amorphous calcium carbonate (ACC).

Calcium carbonate-containing material usable in the context of the present embodiments can be obtained or derived from natural sources (e.g., from living species or geological matter), or be synthetic (chemically synthesized). Commercially available forms of calcium carbonate are also encompassed.

Natural sources of calcium carbonate include, but are not limited to, rock formations, such as limestone, chalk, marble, travertine and tufa, as well as other geological matters. Calcium carbonate is also a principle structural component of many life forms, and thus can be obtained from, inter alia, corals, plankton, coralline algae, sponges, brachiopods, echinoderms, bryozoa, mollusks and other calcium carbonate-containing organisms.

In some of any of the embodiments described herein, the calcium carbonate-containing material comprises one or more forms of crystalline calcium carbonate.

In some of any of the embodiments described herein, the calcium carbonate-containing material comprises, or consists of, one or more forms of aragonite, calcite, ikaite, vaterite, and monohydrocalcite.

In some of any of the embodiments described herein, the calcium carbonate-containing material comprises aragonite. As used herein, the term “aragonite” refers to the crystalline form of calcium carbonate, which can be commonly found in as mineral deposits in caves and in oceans, and in the shells of mollusks and exoskeleton of cold and warm-water corals. The aragonite can be a geological aragonite or a biogenic aragonite (derived from living species such as corals or mollusks). Geological aragonite typically has a crystalline structure that is different from biogenic aragonite.

In some of any of the embodiments described herein, the calcium carbonate-containing material comprises calcite. As used herein, the term “calcite” refers to a crystalline form of calcium carbonate, differing from aragonite in its crystal lattice form, which can be obtained from sedimentary rocks and from the shells of some marine organisms.

In some of any of the embodiments described herein, the calcium carbonate-containing material comprises both aragonite and calcite.

In some of any of the embodiments described herein, the calcium carbonate-containing material (e.g., aragonite) comprises a coral exoskeleton. The term “coral exoskeleton”, as used herein, refers to the exoskeleton of marine madreporic corals or material derived therefrom. Natural coral (e.g., Porites) consists of a mineral phase, principally calcium carbonate, typically in the structural form of aragonite or calcite, with impurities, such as Sr, Mg and F ions, and an organic matrix. Thus, as used herein, “coral exoskeleton” includes calcium carbonate, e.g., in the form of aragonite or calcite, with or without additional components (minerals, organic and inorganic components) derived from or secreted by the living coral or life forms associated therewith.

The term “coral exoskeleton” is also referred to herein simply as “coral skeleton” or abbreviated as “CS”.

In some of any of the embodiments described herein, the calcium carbonate-containing material is derived from a coral and comprises a coral exoskeleton.

Coral exoskeleton can be a commercially available material (e.g., Biocoral™) and has been reported to be biocompatible and resorbable. Coral-derived material described as coralline HA prepared by hydrothermally converting the original calcium carbonate of the coral Porites in the presence of ammonium phosphate, maintaining the original interconnected macroporosity of the coral, is also commercially-available (Pro Osteon®, Interpore Cross). The high content calcium carbonate coral exoskeleton has been shown to be biocompatible and biodegradable at variable rates depending on porosity, the implantation site and the species.

In some of any of the embodiments described herein, the coral exoskeleton or materials comprising the same are derived from a coral. In some embodiments, the coral can comprise any species, including, but not limited to, Porites, Stylophora, Acropora, Millepora, or a combination thereof.

In some embodiments, the coral is from the Porites species. In some embodiments, the coral is Porites Lutea.

In some embodiments, the coral is from the Acropora species. In some embodiments, the coral is Acropora grandis (which in one embodiment is very common, fast growing, and easy to culture). Acropora samples can be easily collected in sheltered areas of the coral reefs and/or can conveniently be cultured.

In some embodiments, the coral is from the Millepora species. In one embodiment, the coral is Millepora dichotoma. In one embodiment, the coral has a pore size of 150 microns and can be cloned and cultured, making Millerpora useful in the compositions and methods of this invention.

In some embodiments, the coral is from the Stylophora species. Stylophora is a genus of colonial stony corals in the family Pocilloporidae, commonly known as cat's paw corals or birdsnest corals. In some embodiments, the coral is Stylophora subseriata.

In another embodiment, the coral can be from any one or more of the following species: Favites halicora; Goniastrea retiforrnis; Acanthastrea echinata; Acanthastrea hemprichi; Acanthastrea ishigakiensis; Acropora aspera; Acropora austera; Acropora sp. “brown digitate”; Acropora carduus; Acropora cerealis; Acropora chesterfieldensis; Acropora clathrata; Acropora cophodactyla; Acropora sp. “danai-like”; Acropora divaricata; Acropora donei; Acropora echinata; Acropora efflorescens; Acropora gemmifera; Acropora globiceps; Acropora granulosa; Acropora cf hemprichi; Acropora kosurini; Acropora cf loisettae; Acropora longicyathus; Acropora loripes; Acropora cf lutkeni; Acropora paniculata; Acropora proximalis; Acropora rudis; Acropora selago; Acropora solitaryensis; Acropora cf spicifera as per Veron; Acropora cf spicifera as per Wallace; Acropora tenuis; Acropora valenciennesi; Acropora vaughani; Acropora verrniculata; Astreopora gracilis; Astreopora rnyriophthalrna; Astreopora randalli; Astreopora suggesta; Australornussa rowleyensis; Coscinaraea collurnna; Coscinaraea crassa; Cynarina lacryrnalis; Distichopora violacea; Echinophyllia echinata; Echinophyllia cf echinoporoides; Echinopora gemmacea; Echinopora hirsutissima; Euphyllia ancora; Euphyllia divisa; Euphyllia yaeyamensis; Favia rotundata; Favia truncatus; Favites acuticollis; Favities pentagona; Fungia granulosa; Fungia klunzingeri; Fungia rnollucensis; Galaxea acrhelia; Goniastrea edwardsi; Goniastea minuta; Hydnophora pilosa; Leptoseris explanata; Leptoseris incrustans; Leptoseris mycetoseroides; Leptoseris scabra; Leptoseris yabei; Lithophyllon undulaturn; Lobophyllia hemprichii; Merulina scabricula; Millepora dichotoma; Millepora exaesa; Millipora intricata; Millepora murrayensis; Millipore platyphylla; Monastrea curta; Monastrea colernani; Montipora caliculata; Montipora capitata; Montipora foveolata; Montipora meandrina; Montipora tuberculosa; Montipora cf vietnamensis; Oulophyllia laevis; Oxypora crassispinosa; Oxypora lacera; Pavona bipartita; Pavona venosa; Pectinia alcicornis; Pectinia paeonea; Platygyra acuta; Platygyra pini; Platygyra sp “green”; Platygyra verweyi; Podabacia cf lanakensis; Porites annae;

Porites cylindrica; Porites evermanni; Porites monticulosa; Psammocora digitata; Psammocora explanulata; Psammocora haimeana; Psammocora superficialis; Sandalolitha dentata; Seriatopora caliendrum; Stylocoeniella armata; Stylocoeniella guentheri; Stylaster sp.; Tubipora musica; Turbinaria stellulata; Stylophora contorta; Stylophora danae; Stylophora kuehlmanni; Stylophora madagascarensis; Stylophora mamillata; Stylophora pistillata; Stylophora subseriata; Stylophora wellsi, or any coral known in the art, or a combination thereof.

Coral exoskeleton is typically porous. In some embodiments, the calcium carbonate-containing material comprises coral exoskeleton having an average pore size (e.g., average diameter) in the range of from 1 micron to 1 millimeter. In one embodiment, the average pore size of a coral ranges from 1 to 200 microns. In one embodiment, the average pore size of a coral ranges from 30 to 180 microns. In one embodiment, the average pore size of a coral ranges from 50 to 500 microns. In one embodiment, the average pore size of a coral ranges from 150 to 220 microns. In one embodiment, the average pore size of a coral ranges from 250 to 1000 microns.

For most therapeutic applications, it is desirable that the calcium carbonate-containing material, when derived from natural sources, such as coral, be devoid of any cellular debris or other organisms associated therewith in its natural state. Thus, in some of any of the embodiments described herein, the coral exoskeleton is an acellular coral exoskeleton.

Calcium carbonate-containing material such as, for example, aragonite, may be a commercially-available material or can be prepared from coral or coral fragments, or from coral sand. Briefly, the coral can be prepared as follows: in one embodiment, coral or coral sand is purified from organic residues, washed, bleached, frozen, dried, sterilized and/or a combination thereof prior to use in the compositions and/or methods of the present embodiments.

In some of any of the embodiments described herein, preparation of the aragonite or coral exoskeleton includes contacting solid coral exoskeleton of a desired size and shape with a solution comprising an oxidizing agent and washing and drying the solid material.

In some of any of the embodiments described herein, the oxidizing agent may be any suitable oxidizing agent, which facilitates the removal of organic debris from the coral exoskeleton. In some embodiments, the oxidizing agent is sodium hypochlorite.

According to this aspect, and in some embodiments, the process comprises conducting said contacting under mildly acidic conditions, so as to remove organic residues and provide acellular coral exoskeleton.

The calcium carbonate-containing material according to some embodiments of the present invention can be provided in a variety of forms, shapes and structures, compatible with a desired application. Some suitable forms and shapes include, but are not limited to, layers, blocks, spherical and hollow spherical forms, concentric spheres, rods, sheets, symmetrical and asymmetrical forms, amorphous and other irregular shapes and particles. The calcium carbonate-containing material can be shaped, for example, to fit a cavity or surface of tissue, or to fit an article containing the composition as described in further hereinafter.

In some of any of the embodiments described herein, the calcium carbonate-containing material (according to any of the respective embodiments and any combination thereof) is provided as particulate calcium carbonate-containing material.

In some embodiments, the particulate material comprises particles having an average particle diameter in the range of from 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns, including any subranges and intermediate values therebetween.

In some of any of the embodiments described herein, a calcium carbonate-containing material is produced from coral or coral sand according to a process comprising washing ground solid calcium carbonate (e.g. aragonite), such as coral or naturally occurring coral sand with water to desalinate it, then disinfecting and drying the desalinated coral sand at temperatures of about 80 degrees to about 150 degrees C., preferably 90 degrees to 120 degrees C., cutting larger pieces of coral into small pieces, and grinding the disinfected and dried coral or coral sand into particles of a desirable average size. In some embodiments, grinding is into particles of a size ranging from 5 to 500 microns.

In some embodiments, coral is ground into particles having a particle diameter of in the range of 1-5, 1-20, 1-50, 1-100, 5-10, 10-15, 15-20, 10-50, 10-100, 20-100, 50-100, 80-150, 100-200, 100-350, 150-500, 1-50 and/or 50-200 microns across, including any intermediate values and subranges therebetween. In some of any of these embodiments, coral is ground to particles having an average particle volume in the range of 1-100, 50-500, 250-1000, 500-2500, 1000-5000 and 2500-10,000 cubic micron or 0.01-0.1, 0.05-0.5, 0.5-0.75, 0.75-1.0, 1.0-2.0 and 1.0-5.0 cubic millimeters in volume, including any intermediate values and subranges therebetween.

In some of any of the embodiments described herein, the calcium carbonate-containing particulate material, including ACC and/or CS and/or any other material as described herein, comprises particles having a relatively small average particle diameter, for example, in a range of from 0.1 micron to 100 microns, or from 0.1 microns to 50 microns, including any intermediate values and subranges therebetween.

In some of any of the embodiments described herein, the calcium carbonate-containing particulate material, including ACC and/or CS and/or any other material as described herein, comprises particles having a relatively large average particle diameter, for example, an average diameter higher than 50 microns, for example, in the range of from 50 microns to 10 millimeter, or from 50 microns to 1 millimeter, or from 100 microns to 1 millimeter.

In some of any of the embodiments described herein, the calcium carbonate-containing particulate material, including ACC and/or CS and/or any other material as described herein, comprises a mixture of particles having a relatively large average particle diameter, as described herein, and particles having a relatively small average particle diameter, as described herein.

Exemplary calcium carbonate-containing materials are described in the Examples section that follows.

The Citrate:

As used herein, the term “citrate salt” describes a compound composed of a citrate ion and one or more cations. The citrate ion can be represented by the formula C₆H₅O₇³⁻ or C₃H₅₀(COO)₃³⁻. The cation can be monovalent, divalent or trivalent cation, and the stoichiometry of the citrate ion is in accordance with the selected cation.

The cation can be Na⁺, K⁺, Li⁺, Mg⁺², Zn⁺², Fe⁺², Fe⁺³, and any other suitable cation. If the cation is a monovalent cation, such as, for example, sodium cation, the citrate salt comprises 3 cations, and is, for example, tri-sodium citrate.

In some of any of the embodiments described herein, other salts of multicarboxylic acids can be used as alternative, or in addition, to a citrate salt as described herein.

By “multicarboxylic acid” it is meant an organic compound featuring two, three or more carboxylic acid groups. For a non-limiting example, a multicarboxylic acid can be represented by R(COOH)n, with R being an alkyl, alkenyl, cycloalkyl, and/or aryl, and n being an integer of at least 2 (e.g., 2, 3, 4, 5, etc.). The alkyl, alkenyl, cycloalkyl, or aryl, can be further substituted by one or more other substituents, as described herein.

In some of any of the embodiments described herein, other calcium-chelating agents can be used as alternative, or in addition, to a citrate salt as described herein.

In some of any of the embodiments described herein, other anti-coagulants can be used as alternative, or in addition, to a citrate salt as described herein. In some embodiments, such anti-coagulants are those acting by effecting the formation of cross-linked fibrin. In some embodiments, such anti-coagulants are not acting by effecting platelet aggregation. In some embodiments, the anti-coagulant is other than heparin or similarly-acting anti-coagulants that effect platelet aggregation.

The Associating Moiety:

The associating moiety is aimed at associating the calcium carbonate-containing material and the citrate so as to form a composite material.

As discussed hereinabove, and without being bound to any particular theory, it is assumed that the associating moiety is such that can form physical and/or chemical interactions with one or both of the calcium carbonate-containing material and the citrate.

In some embodiments, the associating moiety is such that can form electrostatic interactions with one or more of the calcium carbonate-containing material and/or the citrate.

In some embodiments, the associating moiety features functional groups that are positively charged or negatively charged at physiological pH. Positively charged groups can form electrostatic interactions with the citrate and/or the carbonate, the latter leading to release of calcium ions. Negatively charged groups can for electrostatic interactions with calcium ions.

In some of any of the embodiments described herein, the associating moiety comprises one or more positively charged groups, and may further comprise one or more negatively charged groups.

According to some of any of the embodiments described herein, the association moiety is a biocompatible moiety.

In exemplary embodiments, the associating moiety is a positively-charged moiety at physiological pH.

The associating moiety can be a polymeric moiety or a non-polymeric moiety.

In some embodiments, the associating moiety is a polymeric moiety.

The polymeric moiety can be a large polymeric moiety, having a molecular weight higher than 100 kDa, or higher than 200 kDa, or 300 kDa or higher than 300 kDa, for example, in a range of from about 100 to about 1000, or from about 200 to about 1000, or from about 300 to about 1000, or from about 300 to about 800, or from about 300 to about 600, kDa, including any intermediate values and subranges therebetween.

The polymeric moiety can be a large polymeric moiety, having a molecular weight of 100 kDa or lower, for example, in a range of from about 10 to about 100, or from about 20 to about 100, or from about 30 to about 100, or from about 30 to about 80, or from about 30 to about 60, kDa, including any intermediate values and subranges therebetween.

Whenever a molecular weight or MW is described herein in the context of polymeric moieties (e.g., polypeptides), it is meant an average molecular weight, typically determined by conventional methods known in the art and/or in accordance with an information provided by the vendor thereof.

In some of any of the embodiments described herein, the associating moiety is a polymeric moiety, preferably a biocompatible polymeric moiety. In exemplary embodiments, the associating moiety is a polypeptide, featuring high or low molecular weight as described herein.

The term “polypeptide” as used herein encompasses native peptide macromolecules, including degradation products, synthetically prepared peptides and recombinant peptides (e.g., recombinantly expressed in a microorganism), as well as peptidomimetic macromolecules (typically, synthetically synthesized peptides), as well as peptoid and semipeptoid macromolecules which are peptide analogs, which may have, for example, modifications rendering the polypeptides more stable. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein below.

Peptide bonds (—CO—NH—) within the polypeptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g. methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), retro-amide bonds (—NH—CO—), peptide derivatives (—N(R)—CH₂—CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some of any of the embodiments described herein may also include one or more modified amino acids or one or more non-amino acid monomers e.g. fatty acids, complex carbohydrates, etc.

The term “amino acid” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables B and C below list naturally occurring amino acids (Table B) and non-conventional or modified amino acids e.g. synthetic (Table C) which can be used with some embodiments of the invention.

TABLE B

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE C

| Non-conventional | | Non-conventional | |
|---|---|---|---|
| amino acid | Code | amino acid | Code |
| Ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Deys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nrnhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmom | L-N-methylornithine | Nmom |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| Penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmom | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline-3- | Tic |
| ethylamino)cyclopropane | | carboxylic acid | |
| Phosphoserine | pSer | phosphothreonine | pThr |
| phosphotyrosine | pTyr | O-methyl-tyrosine | |
| 2-aminoadipic acid | | hydroxylysine | |

The polypeptides of some embodiments of the invention are preferably utilized in a linear form, although it will be appreciated that in cases where cyclization does not severely interfere with polypeptide characteristics, cyclic forms of the polypeptide can also be utilized.

The polypeptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing polypeptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after de-protection, a pentapeptide and so forth. Further description of polypeptide synthesis is disclosed in U.S. Pat. No. 6,472,505. Large scale polypeptide synthesis is described, for example, by Andersson [Biopolymers 2000; 55(3):227-50].

Polypeptides of the present invention can be purified using a variety of standard protein purification techniques, such as, but not limited to, heat treatments, salting out for example with ammonium sulfate, polyethyleneimines (PEI) precipitation, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to some of any of the embodiments described herein, the associating moiety is a polypeptide, as described herein, and the polypeptide comprises one or more amino acid residue(s) that is/are positively charged at physiological pH.

Such amino acid residues typically comprise a primary or secondary amine group at the side chain thereof, and include, for example, the naturally occurring L-lysine, L-arginine, and L-histidine, and non-naturally occurring amino acid analogs thereof, such as, for example, D-lysine, D-arginine, D-histidine, c-Lysine and ornithine.

In some of these embodiments, at least 5%, or at least 10%, or at least 20%, or at least 30%, or at least 40%, or at least 50%, or more, or substantially all, of the amino acid residues in the polypeptide are positively charged at physiological pH. The positively charged amino acid residues can be dispersed randomly within the polypeptide, and can include one or more types of positively charged amino acid residues, such as described herein.

In some of these embodiments, the polypeptide is consisted of amino acid residues that are positively charged amino at physiological pH, and can be, for example, polylysine, polyhistidine, polyarginine, polyornithine, etc.

In exemplary embodiments, the polypeptide comprises a plurality (e.g., at least 20%, or at least 50%, or at least 80%, or 100%) of lysine residues, which can be L-lysine residues and/or D-lysine residues. Alternatively, or in addition, the lysine residues are c-lysine residues.

In exemplary embodiments, the polypeptide comprises a plurality (e.g., at least 20%, or at least 50%, or at least 80%, or 100%) of arginine residues, which can be L-arginine residues and/or D-arginine residues.

In exemplary embodiments, the polypeptide comprises a plurality (e.g., at least 20%, or at least 50%, or at least 80%, or 100%) of hisitine residues, which can be L-histidine residues and/or D-histidine residues.

In exemplary embodiments, the polypeptide comprises a plurality (e.g., at least 20%, or at least 50%, or at least 80%, or 100%) of ornithine residues, which can be L-ornithine residues and/or D-ornithine residues.

In exemplary embodiments, the polypeptide is poly-D-lysine (PDL).

In exemplary embodiments, the polypeptide is poly-L-lysine (PDL).

Other polypeptides are also contemplated, for example, collagen (e.g., Types I, II and III), preferably human collagen, which can be synthetically prepared, recombinant, or extracted from a natural source. In exemplary embodiments, the collagen has average molecular weight (MW) that ranges from about 100 to about 200 kDa.

Any of the polypeptides described herein can have a low or high molecular weight as described herein.

According to some of any of the embodiments described herein, the associating moiety is a non-polymeric moiety.

The non-polymeric moiety can be, for example, positively charged at physiological pH.

The non-polymeric moiety can be, for example, an amino acid that is positively charged at physiological pH, as described herein in any of the respective embodiments.

In exemplary embodiments, the amino acid is L-lysine and/or D-lysine.

In exemplary embodiments, the amino acid is arginine, histidine, ornithine or c-lysine.

In some of any of the embodiments described herein, the associating moiety is selected as capable of affecting blood coagulation in a subject. Affecting blood coagulation can be by promoting or participating in one or more pathways that are associated with blood coagulation (see, for example, FIG. 1).

In some embodiments, the associating moiety is capable of interfering in (e.g., inhibiting) a fibrinolysis process in a subject. Exemplary such associating moieties are positively charged polymeric and/or non-polymeric moieties as described herein (e.g., polypeptides and/or amino acids), and/or moieties that structurally resemble coagulants such as tranexamic acid or aminocaproic acid.

Without being bound by any particular theory, it is assumed that lysine, by sharing structural features with tranexamic acid, either as an amino acid or as part of a polypeptide as described herein, can inhibit fibrinolysis and thereby promote blood coagulation.

According to exemplary embodiments, the associating moiety is poly-D-lysine, poly-L-lysine, poly-D-L-lysine (with any ratio of the D-lysine and L-lysine), each having a high or low molecular weight as described herein, or any mixture thereof.

According to exemplary embodiments, the associating moiety is D-lysine, L-lysine, or a mixture thereof.

The Composite Material:

The composite material of the present embodiments comprises the citrate, calcium-carbonate-containing material and the associating moiety, associated to one another, and encompasses any form of association, as described herein, between these components, and at any order.

In an exemplary configuration, at least a portion of the association moiety is deposited onto at least a portion of the surface of the calcium carbonate-containing material. In some of these embodiments, the calcium carbonate-containing material is a particulate material and the association moiety is deposited on a portion of the surface or practically coats the surface of at least a portion or all of the calcium carbonate-containing material particles.

In some of these embodiments, at least a portion of the calcium carbonate-containing material is associated with at least a portion of the association moiety via electrostatic interactions formed between the carbonate of the calcium carbonate-containing material and a positively charged group of the association moiety.

Further in this exemplary configuration, at least a portion of the citrate is associated with the portion of association moiety which is deposited onto a surface of the particulate material. Alternatively, or in addition, the citrate is associated with the calcium carbonate-containing material as described herein.

In some of these embodiments, at least a portion of the citrate is associated with at least a portion of the association moiety via electrostatic interactions and/or hydrogen bond interactions.

In some of any of the embodiments described herein, a weight ratio of the citrate and the calcium carbonate-containing material ranges from 10:1 to 1:10, or from 5:1 to 1:5, including any intermediate values and subranges therebetween, and can be, for example, 10:1, 5:1, 2:1, 1:1, 1:2, 1:5 or 1:10.

In some of any of the embodiments described herein, a weight ratio of the associating moiety and the calcium carbonate-containing material ranges from 5000:1 to 250:1, or from 2500:1 to 250:1, including any intermediate values and subranges therebetween, and can be, for example, 5000:1, 2500:1, 1000:1, 500:1 or 250:1.

The composite material may further comprise one or more additional components which may improve its function. Exemplary such components are swelling or thickening agents, for example, polymeric materials or moieties such as, but not limited to, polysaccharides (e.g., alginate, chitosan), poly (alkylene glycols) (e.g., poly (ethylene glycol)) and/or polypeptides such as collagen. Such polymeric materials may act as strong absorbents, binding to platelets and/or other components of the coagulation cascade, locally enhancing formation of blood clots.

Exemplary such components are coagulating agents (pro-coagulants) such as, but not limited to, fibrinogen, thrombin and/or plasminogen.

Exemplary such components include blood tissue or cells, preferably dried blood tissue or cells.

Preferably, the additional components are selected such as they do not interfere with the association and performance of the citrate, the calcium carbonate-containing material and the associating moiety.

In exemplary embodiments, the composite material may further comprise collagen, which is in association with the calcium carbonate, the associating moiety and/or the citrate.

According to an aspect of some embodiments of the present invention there is provided a composite material that comprises a calcium carbonate-containing material as described herein in any of the respective embodiments and any combination thereof, and an associating moiety as described herein in any of the respective embodiments and any combination thereof.

### Compositions:

The composite material described herein can be used per se, or can be formulated together with a pharmaceutically acceptable carrier, to form a composition, e.g., a pharmaceutical composition.

As used herein, the term “pharmaceutically acceptable carrier” describes a carrier or a diluent that is used to facilitate the administration of the composite material (also referred to in this context as an active ingredient or active agent) or of the composition containing same and which does not cause significant irritation to an organism and does not abrogate the biological activity and properties of the administered active materials. Examples, without limitations, of carriers include water, buffered aqueous solutions, propylene glycol, emulsions and mixtures of organic solvents with water, as well as solid (e.g. powdered or polymeric) and gaseous carriers.

Techniques for formulation and administration of drugs may be found in “Remington's Pharmaceutical Sciences” Mack Publishing Co., Easton, Pa., latest edition, which is incorporated herein by reference.

Compositions for use in accordance with the present embodiments thus may be formulated in conventional manner using one or more pharmaceutically acceptable carriers, excipients and/or auxiliaries, which facilitate processing of the compounds into preparations which can be used pharmaceutically. The dosage may vary depending upon the dosage form employed and the route of administration utilized.

The exact formulation, route of administration and dosage can be chosen by the individual physician in view of the patient's condition (see e.g., Fingl et al., 1975, in “The Pharmacological Basis of Therapeutics”, Ch. 1 p.1).

The pharmaceutically acceptable carrier can be either an organic carrier or an aqueous carrier. In some embodiments, the carrier is an aqueous carrier. An aqueous carrier preferably comprises injectable-grade water, i.e., USP grade “water for injection”. However, other forms of purified water may be suitable, such as, for example, distilled and deionized water.

Aqueous formulations are preferred since these formulations are gentle to bodily tissues and are suitable for use on injured blood vessels or tissues. However, non-aqueous formulations are also contemplated. For example, in cases where the composition is in a form of a paste or an emulsion, non-aqueous carriers or mixed carriers of aqueous and organic carriers can be used.

The composition may be formulated for administration in either one or more of routes, depending on the area to be treated.

According to some embodiments, the composition is formulated for topical application, as a topical dosage form.

As used herein, the phrase “topical dosage form” describes a dosage form suitable for topical administration to the treated area (e.g., an injured blood vessel or tissue). By “topical administration” it is meant application onto the treated area, or “local administration”, whereby the treated area can be, for example, an internal or external injured blood vessel or tissue.

The compositions described herein can be, for example, in a form of a powder, granules, a cream, an ointment, a paste, a gel, a lotion, a milk, a suspension, an aerosol, a spray, a foam, a gauze, a wipe, a sponge, a wound dressing, a pledget, a patch, a pad, an adhesive bandage, and a non-adhesive bandage.

In some embodiments, the composition is formulated as a liquid reservoir, to be applied as drops, spray, aerosol, liquid, foam and the like. Suitable carriers and other ingredients are used in these cases. For example, for application as an aerosol or foam, a propellant is used. For application as foam, foam-forming agents can also be used.

In some embodiments, the composition is formulated as a cream. Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information. An exemplary cream formulation can be obtained by mixing the composite material described herein with a carrier comprising cellulose derivatives such as cellulose acetate, hydroxyethyl cellulose and/or a polyethylene glycol. In some embodiments, the composition is formulated as an ointment. Ointments are semisolid preparations, typically based on petrolatum or petroleum derivatives. The specific ointment base to be used is one that provides for optimum delivery for the active agent chosen for a given formulation. As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

In some embodiments, the composition is formulated as a lotion. Lotions are preparations that are to be applied to the skin surface without friction. Lotions are typically liquid or semiliquid preparations in which solid particles, namely, the calcium carbonate-containing material particles, are present in a water or alcohol base. Lotions are typically preferred for covering/protecting large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

In some embodiments, the composition is formulated as a paste. Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

In some embodiments, the composition is formulated as a gel. Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

In some embodiments, the composition is formulated as a foam. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the treated area, quickly evaporates, driving the composite material to the site of treatment.

In some embodiments, the composition is formulated as a powder or granules. Such compositions can optionally be prepared by preparing the composite material (e.g., as described herein) and forming granules or beads containing these ingredients, for example, by adding suitable agents (e.g., water soluble film-forming agents).

In some embodiments, a topical dosage form includes a solid or semi-solid substrate, e.g., a gauze, a wipe, a bandage, a pad, a pledget, a sponge, a mesh, a fabric, and the likes, and the composite material is incorporated in and/or on the substrate.

The substrate in such topical dosage forms can be of any form and materials used to make up gauzes, wipes, bandages, pads, pledgets, sponges, meshes, fabrics (woven and non-woven, cotton fabrics, and the like), and any other substrates commonly used in medical applications.

Such topical dosage forms may optionally further comprise an adhesive, for facilitating the topical application of the composition onto the treated area for a prolonged time period.

Exemplary adhesives include, but are not limited to, medically acceptable bioadhesives, polymer glues, etc., and can be applied to the substrate by, for example, dip coating with an adhesive base. Such dip coating can be effected during manufacture of the substrate, or at any time prior to its application. In some embodiments, the composite material can be embedded within and/or on the material of the substrate, for example, embedded into or onto a polymer or fabrics by application of heat, or fused to the substrate. In other embodiments, the composite material can be incorporated into the base material of the substrate, for example, mixed within the components of a polymer before polymerization, or mixed with components forming fibers used to make up a gauze or a mesh or pad, etc.

In some of any of the embodiments described herein, the composition is also referred to as an anti-hemorrhaging composition.

The composition described herein can further comprise additional ingredients, which are aimed at improving or facilitating its preparation, application and/or performance. Such additional ingredients include, for example, anti-irritants, anti-foaming agents, humectants, deodorants, antiperspirants, preservatives, emulsifiers, occlusive agents, emollients, thickeners, penetration enhancers, colorants, propellants and/or surfactants, depending on the final form of the composition.

Representative examples of humectants that are usable in this context of the present embodiments include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Representative examples of deodorant agents that are usable in the context of the present embodiments include, without limitation, 2,4,4′-trichloro-2′-hydroxy diphenyl ether, and diaminoalkyl amides such as L-lysine hexadecyl amide.

Suitable preservatives that can be used in the context of the present embodiments include, without limitation, one or more alkanols, parabens such as methylparaben and propylparaben, propylene glycols, sorbates, urea derivatives such as diazolindinyl urea, or any combinations thereof.

Suitable emulsifiers that can be used in the context of the present embodiments include, for example, one or more sorbitans, alkoxylated fatty alcohols, alkylpolyglycosides, soaps, alkyl sulfates, or any combinations thereof.

Suitable occlusive agents that can be used in the context of the present embodiments include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, that can be used in the context of the present embodiments include, for example, dodecane, squalane, cholesterol, isohexadecane, isononyl isononanoate, PPG ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners that can be used in the context of the present embodiments include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, and mixtures thereof.

Suitable penetration enhancers usable in context of the present embodiments include, but are not limited to, polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, menthol, TWEENS such as TWEEN 20, and the like. The permeation enhancer may also be a vegetable oil. Such oils include, for example, safflower oil, cottonseed oil and corn oil.

Suitable anti-irritants that can be used in the context of the present embodiments include, for example, steroidal and non-steroidal anti-inflammatory agents or other materials such as menthol, aloe vera, chamomile, alpha-bisabolol, cola nitida extract, green tea extract, tea tree oil, licorice extract, allantoin, caffeine or other xanthines, glycyrrhizic acid and its derivatives.

Any of the additional ingredients or agents described herein is preferably selected as being compatible with the components of the composite material as described herein, such that there is no interference with the availability of these materials in the composition.

Any of the additional ingredients described herein is further preferably selected as being biocompatible.

In some embodiments, the composition further comprises an additional therapeutically active agent, for example, an additional hemostatic agent or composition or article, or, for example, an agent capable of disinfecting the treated area (e.g., antiseptic agents or compositions).

Compositions of the present embodiments may, if desired, be presented in a pack or dispenser device, such as an FDA (the U.S. Food and Drug Administration) approved kit, which may contain one or more unit dosage forms containing the composite material. The pack may comprise, for example, glass or plastic foil. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions of the invention formulated in a compatible pharmaceutical carrier may also be prepared, placed in an appropriate container, and labeled for a medical indication, as detailed herein.

The compositions described herein may be packed or presented in any convenient way. For example, they may be packed in a tube, a bottle, a dispenser, a squeezable container, or a pressurized container, using techniques well known to those skilled in the art and as set forth in reference works such as Remington's Pharmaceutical Science 15^{th} Ed. It is preferred that the packaging is done in such a way so as to minimize contact of the unused compositions with the environment, in order to minimize contamination of the compositions before and after the container is opened.

The compositions described herein are preferably supplied in the concentration intended for use but may also be prepared as concentrates that are diluted prior to use. For example, concentrates requiring dilution ratios of 2:1 to 10:1 parts carrier to a concentrate are contemplated.

In some embodiments, the composition described herein is packaged in a packaging material and identified in print, in or on the packaging material, for use in inducing blood coagulation and/or in reducing or arresting hemorrhaging, as described herein.

Articles-of-Manufacturing:

According to an aspect of some embodiments of the present invention, an article-of-manufacturing is provided, which comprises the composite material or the composition as described herein in any of the respective embodiments, and any combination thereof, and means for topically applying the composite material or a composition comprising same onto the treated area. In some embodiments, the article-of-manufacturing is configured to apply the composition to an injured blood vessel or tissue.

In some embodiments, the article-of-manufacturing comprises the composition as described herein, in a form of a suspension, packaged in a container, and means for applying the composition as drops, spray, aerosol, foam, using techniques well known to those skilled in the art and as described herein.

In some embodiments, the article-of-manufacturing comprises the composition as described herein, in a form of a cream, lotion, paste, ointment, and the likes, packaged in a suitable container, and optionally comprising means for dispensing the composition from the container.

In some embodiments, the article-of-manufacturing comprises the composite material or a composition comprising same as described herein, in a form that comprises a powder or granules, packaged in a suitable container, and optionally comprising means for dispensing the composition from the container.

In some embodiments, the article-of-manufacturing comprises the composite material or a composition comprising same as described herein, incorporated in and/or on a substrate, as described herein. The article-of-manufacturing can be packaged in a sterile packaging. In exemplary embodiments, the substrate is a gauze or any other solid substrate usable in medical applications, and the article-of-manufacturing is bandage comprising the composite material.

The article-of-manufacturing can be labeled as described herein, for example, by being identified in print, in or on the packaging material, for use in inducing blood coagulation and/or reducing or arresting hemorrhaging, or in any other use, as described herein.

Kits:

According to an additional aspect of embodiments of the invention there is provided a kit, which comprises the composite material or a composition comprising same as described herein, being packaged in a packaging material.

The kit can be labeled, for example, by being identified in print, in or on the packaging material, for use in inducing blood coagulation and/or reducing or arresting hemorrhaging, as described herein.

The components of the composition can be packaged within the kit either together, as a single, ready for use, composition, or at least one of the components (e.g., a carrier or a solid substrate) can be packaged individually. When one or more components are packaged individually, the kit may further be supplied with instructions indicating the route of preparing an anti-hemorrhaging composition, or otherwise indicating how to apply the components so as to contact an area to be treated with the composite material or the composition. Such instructions can be, for example, mixing the components (e.g., the composite material and the carrier) prior to application or simultaneously or subsequently applying the components (e.g., the composite material and the carrier) onto an area to be treated.

In an exemplary embodiment, the kit comprises the composite material in a first container (e.g., a sterile packaging), and a liquid carrier packaged individually (e.g., in another container), and instructions to add the carrier to the first container, prior to application of the composition to the area to be treated, or vice versa (instructions to add the content of the first container to the carrier in the second container). The first (or second) container can be configured to apply the composition as drops, spray, aerosol, foam, etc. The kit may alternatively further comprise a device for dispensing the composition.

In another exemplary embodiment, the kit comprises the composite material in a first container (e.g., a sterile packaging), and a solid carrier (e.g., a gauze) packaged individually (e.g., in another container), and instructions to contact the composite material with the solid carrier to provide a composition, prior to application of the composition to the area to be treated or instructions to contact an injured area to be treated with the composite material and apply the gauze or other solid substrate onto the applied composite material. The kit may alternatively further comprise a device for dispensing the composite material.

In another exemplary embodiment, the kit comprises the calcium carbonate-containing material, the association moiety and the citrate, each packaged individually, and each optionally together with a carrier, or a carrier is optionally packaged individually with the kit. The carrier is such that is suitable for the selected dosage form, as described herein. The kit further comprises instructions to prepare the composite material, as described herein, and optionally mix the composite material with the carrier or otherwise use the composite material in combination with the carrier as described herein.

The containers, substrates, and compositions included in the kit can be in accordance with any of the embodiments described herein, and any combination thereof.

Uses:

As demonstrated in the Examples section that follows, it has been uncovered that a composite material as described herein is capable of inducing blood coagulation and arrest bleeding within a short time period, even in cases of massive hemorrhaging, and that the composite material is (i) convenient for use as it is possible to apply it as is directly on a bleeding tissue or organ; and (ii) exhibits improved performance as compared to each of its components alone and to each pair of components out of the three main components thereof.

As used herein, the term “coagulation of blood” or “blood coagulation” describes clot formation in blood, namely, the formation blood clots in a subject's plasma. The clot formation can result from either or both of the intrinsic cascade, initiated when contact is made between blood and exposed negatively charged surfaces, and the extrinsic pathway, initiated upon vascular injury, leading to activation of factor X to Xa which hydrolyzes and activates prothrombin to thrombin. Thrombin then activates factors XI, VIII and V, until ultimately fibrinogen is converted to fibrin and factor XIII to XIIIa. Factor XIIIa (also termed transglutaminase) cross-links fibrin polymers solidifying the clot. Thus, as used herein, the term “clot” or “thrombus” refers to the final product of the blood coagulation step in hemostasis. There are two components to a clot/thrombus: aggregated platelets that form a platelet plug, and a mesh of cross-linked fibrin protein. The substance making up a thrombus is also known as cruor.

In some embodiments of the present invention, the blood is mammalian blood. In some embodiments, the blood is human blood.

The “inducing coagulation of blood”, as used in the context of embodiments of the present invention, describes inducing coagulation and/or influencing the coagulation state of blood by increasing coagulation or coagulation rate of the blood. As such, the compositions described herein are characterized by increasing the clotting of blood and blood clotting state, which includes increasing clotting of plasma or increasing clotting rate of plasma, as well as reducing or preventing or decreasing a rate of lysis or dissolution of a blood clot.

By “clotting” it is meant formation of blood clots. By “clotting rate” or “coagulation rate”, it is meant the percent of unclotted blood that turns into clotted blood within a certain time frame.

Clotting of blood and/or a rate of blood clots formation can be monitored in a variety of assays known in the art.

Exemplary techniques used in such assays include clot-based tests, chromogenic or color assays, direct chemical measurements, and ELISAs, are used for coagulation testing. An exemplary assay is the aPTT (activated partial thromboplastin time), performed by adding a surface activator (e.g., kaolin, celite, ellagic acid, or silica) and diluted phospholipid (e.g., cephalin) to citrated plasma. After incubation to allow optimal activation of contact factors (factor XII, factor XI, prekallikrein, and high-molecular-weight kininogen), calcium is added, and the clotting time is measured through absorbance. Clot-based assays use mostly citrated plasma, and the end point for all of them is fibrin clot formation.

In some embodiments, the composite material or a composition containing same described herein can reduce the clotting time of human plasma and/or increase the clotting rate of human plasma.

According to some embodiments of the present invention, the composite material or a composition containing same as described herein is capable of inducing blood coagulation (blood clots formation) upon contacting the blood for a time period that ranges from a few seconds to a few minutes, for example, of 10 minutes or less, e.g., 9, 8, 7, 6, 5, 4, 3, 2 minutes or less, or of one minute or less (e.g., 50 seconds, 40 seconds, 30 seconds or less) regardless of the blood's volume.

According to some embodiments of the present invention, the composite material or a composition containing same as described herein is such that upon contacting the blood, e.g., for a time period as described herein, at least 50% of a blood sample turns into blood clots.

According to some embodiments of the present invention, at least 50% of the clotted blood (formed upon contacting the composition as described herein) remains clotted for at least one hour, or for at least 2 hours, or for at least three hours, or for at least 4 hours, or more.

Any of the compositions, articles and kits described herein can therefore be used for inducing blood coagulation in a subject in need thereof.

As used herein, the term “subject” includes mammals, preferably warm-blooded mammals including birds, cows, horses, goat, sheep, pigs, dogs, cats, chickens and turkeys, and more preferably human beings at any age which suffer from a pathology that requires induction of blood coagulation.

According to an aspect of some embodiments of the present invention, there is provided a method of inducing blood coagulation, which is effected by contacting the blood with a composite material or a composition containing same as described herein.

The contacting can be effected in vitro or ex vivo for example, by contacting a blood sample with composite material or a composition containing same as described herein.

The contacting can alternatively be effected in vivo, by contacting a blood of a subject with a composite material or a composition containing same as described herein.

According to an aspect of some embodiments of the present invention, there is provided a use of composite material or of a composition or article-of-manufacturing containing same as described herein for inducing coagulation of blood. Inducing blood coagulation can be effected in vitro, ex vivo, or in vivo, as described herein.

According to an aspect of some embodiments of the present invention there is provided a use of the composite material or a composition or article-of-manufacturing containing same as described herein in the manufacture of a medicament for inducing coagulation of blood in a subject in need thereof.

According to some of any of the embodiments described herein for the methods and uses of the compositions described herein, the contacting is effected such that at least 50% of the blood is clotted upon contacting with the composite material or a composition comprises same for less than 10 minutes, or less than 5 minutes, or less, as described hereinabove.

According to some embodiments of the present invention, the contacting is effected such that at least 50% of the clotted blood formed upon said contacting remains clotted for at least one hour, as described herein.

According to some of any of the embodiments described herein for the methods and uses of the compositions described herein, the contacting is effected by applying composite material or a composition containing same, or an article-of-manufacturing containing same, to an injured blood vessel.

The injured blood vessel can be an internal or external blood vessel, and can form a part of an injured tissue or organ.

The term “tissue” refers to part of an organism consisting of cells designed to perform a function or functions. Examples of tissue include, but are not limited to, skin tissue, hepatic tissue, pancreatic tissue, blood tissue, cardiac tissue, gastrointestinal tissue, vascular tissue, renal tissue, pulmonary tissue, gonadal tissue, hematopoietic tissue, nervous tissue, abdominal tissue, and spleen tissue.

When contacting is effected in vivo, the composite material or a composition containing same is preferably administered in a direct, local manner, for example, via placement or application (e.g., by injection) of the composite material or a composition containing same, or an article-of-manufacturing containing same, directly into or onto an injured tissue region of a subject.

In some of any of the embodiments described herein, contacting blood with the composite material or a composition containing same or an article-of-manufacturing containing same, as described herein, is effected such that an effective amount of the composite material is contacted with the blood (e.g., an injured blood vessel or tissue, also referred to herein as an area to be treated).

By “effective amount” it is meant an amount that induces blood coagulation by turning at least 50% of the blood that contacts the composite material into blood clots within no more than 30 minutes, or no more than 20 minutes, or no more than 10 minutes, or within a shorter time period as described hereinabove.

In exemplary, non-limiting embodiments, an effective amount of a composite material as described herein is 1-20, or 5-20, or 5-10, grams, of the composite material per 1 ml blood that contacts the composite material.

The contacting of the injured blood vessel or tissue with the composite material or a composition containing same or an article-of-manufacturing containing same, can be effected while using any of the compositions, articles-of-manufacturing or kits as described herein, or using the composite material per se.

In some of any of the embodiments described herein, the contacting further comprises applying pressure to the bleeding organ and/or tissue when contacted with the composite material or composition. That is, once the composite material or composition contacts the bleeding organ and/or tissue, pressure is applied to the treated area. In exemplary embodiments, pressure is applied by applying a compressive force. In exemplary embodiments, the compressive force is applied by a human-being, for example, by a force applied by a hand of human of an average size. In exemplary embodiments, the pressure/compressive force is within a range of from 1-3 kg/25 cm².

Generally, pressure or compressive force is applied for a time period at which bleeding is arrested. In some of any of these embodiments, applying the pressure or compressive force is for a time period that ranges from a few seconds to a few minutes, for example, from about 0.1 minute to 10 minutes, or from 0.1 minute to 5 minutes, or from 0.1 minute to 3 minutes, or from 0.1 minute to 2 minutes, or from 0.5 minute to 5 minutes, or from 0.5 minute to 3 minutes, including any intermediate values and subranges therebetween.

Any of the composite materials, compositions, articles, kits, methods and uses described herein for inducing blood coagulation can be efficiently utilized for reducing or arresting hemorrhaging in a subject in need thereof.

The hemorrhaging can be an external or, preferably, an internal hemorrhaging.

Accordingly, the composite materials, compositions or article-of-manufacturing containing same and methods described herein can be used for treating hemorrhaging, e.g., internal hemorrhaging, in a subject in need thereof, by contacting an injured blood vessel or tissue of the subject with a composite material or a composition containing same as described herein.

In some embodiments, the contacting is effected outside a medical facility (e.g., a hospital), for example, at a site where trauma has occurred, as an emergency treatment. In some embodiments, treating hemorrhaging is followed by a surgical procedure, e.g., by procedures well known in the art.

In some embodiments, the contacting is effected during a surgical procedure, to assist in arresting hemorrhaging as a result of a trauma or as a result of the surgical procedure itself.

In some of any of the embodiments described herein, the composite material or composition or article-of-manufacturing containing same is for use in, or is utilized in a method of, reducing or arresting a massive hemorrhaging.

Herein throughout, “massive hemorrhaging” can be defined as meeting one or more of the following: massive bleeding which is such that the blood loss of a subject exceeds the volume of his circulating blood occurs within a 24-hour period; massive bleeding which is such that the blood loss of 50% of the volume of the circulating blood volume occurs within a 3-hour period; bleeding at a rate of 150 ml/minutes or more; and bleeding that results in blood loss that necessitates plasma and platelet transfusion.

In some embodiments, any of the composite materials, compositions, methods and uses described herein for inducing blood coagulation are utilized for inducing blood coagulation in subjects suffering from a disease or disorder in which increasing blood clots formation or increasing a rate of blood coagulation is desired. Such diseases and disorders include, for example, hemophilia, dialysis treatments, damage control during operations and patients using anti-coagulants.

Any of the composite materials, compositions, articles, kits, methods and uses described herein for inducing blood coagulation can be efficiently utilized for treating traumatic brain injury, by arresting associated hemorrhage and by increasing recovery or regeneration of brain functions, as exemplified in the Examples section that follows.

According to an aspect of some embodiments of the present invention, the composite material, or a composition or article-of-manufacturing containing same as described herein in any of the respective embodiments are for use in treating traumatic brain injury.

According to an aspect of some embodiments of the present invention, there is provided a method of treating traumatic brain injury, which comprises contacting the injury site with a composite material, or a composition as described herein in any of the respective embodiments.

The treatment according to some of these embodiments comprises contacting the injured site (in the brain) with the composite material or the composition. The contacting can be transient, for example, as described herein (along with application of pressure) or by implanting the composite material at the injured site.

As demonstrated in the Examples section that follows, such a treatment increases the recovery and/or generation of brain functions following the TBI.

It is expected that during the life of a patent maturing from this application many relevant calcium carbonate-containing materials and/or citrate salts and/or association moieties will be developed and the scope of the terms “carbonate-containing material” and “citrate salt” and “association moiety” are intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Exemplary Calcium Carbonate-Association Moiety-Citrate (CPC) Composites Materials

Trisodium citrate was obtained from Sigma-Aldrich.

7% hypochlorite solution was obtained from CARLO ERBA reagents.

H₂O₂ solution (Gerdrogen 30% by weight) was obtained from Riedel-de Haen, Germany.

Table 1 below presents exemplary calcium carbonate-containing materials that are usable in the context of the present embodiments.

Mixed coral skeleton particles were obtained from several types of aquarium-grown corals, having an average size of 40-300 microns (large particles) or lower than 40 microns (e.g., 0.2-40 microns or 0.2-30 microns; small particles) or a mix of small and large particles (0.1-300 microns; mixed particles). Exemplary mixed coral skeleton particles were obtained from Porites lutea, Trachiphillia, and Stylophora aquarium-grown corals.

Single coral skeleton particles were obtained from one type of an aquarium-grown coral, having an average size of 40-300 microns (large particles) or lower than 40 microns (e.g., 0.2-40 microns or 0.2-30 microns; small particles) or a mix of small and large particles (0.1-300 microns; mixed particles). Exemplary single coral skeleton particles were obtained from aquarium-grown Stylophora.

Herein throughout the term “micron” refers to micrometer or μm.

TABLE 1

| | | Average | | |
|---|---|---|---|---|
| | Vendor | particle | | |
| Material | (Catalog Number) | size | | Remarks |
| Amorphous | Glentham | Up to about 30 | | Abbreviated as |
| calcium | (GK3384) | microns | | ACC |
| carbonate | | | | |
| Geological | Alfa Aesar | >2 | mm | |
| aragonite | (42523) | | | |
| Geological | Alfa Aesar | 20-60 | microns | Obtained by |
| aragonite | (42523) | | | grinding the |
| | | | | particles |
| | | | | sized > 2 mm |
| Mixed Coral | Coral skeleton | 0.2-40 | microns | Abbreviated |
| skeleton | particles obtained | | | mixed CS (small) |
| particles | from several | | | |
| | types (e.g., Porites | | | |
| | lutea, Trachiphillia, | | | |
| | and Stylophora) | | | |
| | aquarium-grown | | | |
| | corals | | | |
| | Coral skeleton | 40-300 | microns | Abbreviated |
| | particles obtained | | | Mixed CS (large) |
| | from several | | | |
| | types (e.g., Porites | | | |
| | lutea, Trachiphillia, | | | |
| | and Stylophora) | | | |
| | aquarium-grown | | | |
| | corals | | | |
| | Coral skeleton | 0.1-300 | microns | Abbreviated |
| | particles obtained | | | Mixed CS (mix) |
| | from several | | | |
| | types (e.g., Porites | | | |
| | lutea, Trachiphillia, | | | |
| | and Stylophora) | | | |
| | aquarium-grown | | | |
| | corals | | | |
| Single Coral | Coral skeleton | 40-300 | microns | Abbreviated |
| Skeleton | particles obtained | | | Single CS (large) |
| particles | from one type (e.g. | | | |
| | Stylophora) of | | | |
| | aquarium-grown | | | |
| | corals | | | |
| Single Coral | Coral skeleton | 0.2-40 | microns | Abbreviated |
| Skeleton | particles obtained | | | Single CS (small) |
| particles | from one type (e.g. | | | |
| | Stylophora) of | | | |
| | aquarium-grown | | | |
| | corals | | | |
| Single Coral | Coral skeleton | 0.1-300 | microns | Abbreviated |
| Skeleton | particles obtained | | | Single CS (mix) |
| particles | from one type (e.g. | | | |
| | Stylophora) of | | | |
| | aquarium-grown | | | |
| | corals | | | |

Table 2 below presents exemplary association moieties that are usable in the context of the present embodiments.

TABLE 2

| | Molecular weight | | Vendor and catalog |
|---|---|---|---|
| Material | MW | | number |
| Poly-D-lysine | 30000-70000 | Da | Sigma P7280\P7786 |
| | 1000-5000 | Da | Sigma P0296 |
| | 4000-15000 | Da | Sigma P6403 |
| | >300,000 | Da | A003E Mercury |
| Poly-L-lysine | 1000-4000 | | Sigma P0879 |
| | 4000-15000 | | Sigma P6516 |
| | 15000-30000 | | Sigma P7890 |
| | 30,000-70,000 | Da | Sigma P2636 |
| | 150000-300000 | Da | Sigma P4382 |
| Poly-ε-lysine | 500-4000 | Da | Sigma P4510 |
| L-lysine | 146.19 | Da | sigma L5501 |
| D-lysine | 146.19 | Da | Sigma L-8021 |
| Poly-L-arginine | 5000-15,000 | Da | Sigma P4663 |
| | 50,000-70,000 | Da | Sigma P7762 |
| | >70,000 | Da | Sigma P3892 |
| L-arginine | | | Sigma 5006 |
| D-arginine | | | Sigma 2646 |
| Poly-L-histidine | >5000 | Da | Sigma P2534 |
| L-histidine | | | Sigma H8000 |
| D-histidine | | | Sigma H3751 |
| Poly-ornithine | 5,000-15,000 | Da | Sigma P4538 |
| | 15,000-30,000 | Da | Sigma P2533 |
| | 30,000-60,000 | Da | Sigma P3655, P4597 |
| | >100,000 | Da | Sigma P4638 |

| | | |
|---|---|---|
| Collagen | about 140,000 Da | Mercury Type: |
| | | 1rat tail 08-115 |

Table 3 below presents additional materials that may be added to the composites of the present embodiments:

TABLE 3

| | Molecular weight | |
|---|---|---|
| Material | MW | Vendor and catalog number |
| Coagulants: | | |
| Thrombin | Human plasma | Mercury 605190-100U |
| Fibrinogen | Human plasma | Mercury 3169957 |
| Swelling agents: | | |
| Alginate | 405.2 Da | Sigma W201502 |
| Poly-ethylene-glycol | 6,000 Da | |
| Collagen | about 140,000 Da | Mercury Type: 1rat tail 08-115 |
| Chitosan | 5,000 Da | Sigma Cat. # 448869 |

The addition of coagulants enhances the effect imparted by the composite material in arresting hemorrhage. The addition of swelling agents results in thickening of the blood and increase in its viscosity, which should promote and/or stabilize blood coagulation.

Preparation of an Exemplary CPC Conjugate:

Poly-D-lysine (PDL) having MW of 30-70 kDa was selected as an exemplary polymeric unit linking a citrate salt and a calcium carbonate-containing material to one another.

This exemplary polymeric conjugate was prepared as follows:

Coral skeleton (from a single coral type as described in Table 1 above) pieces sized 0.5-1 cm in length were cleaned from organic residues by exposure to three solutions in a raw: 10% hypochloric acid, 1 M NaOH, and 30% hydrogen peroxide solution, as described, for example, in Weiss et al., 2019, J Biomed Mater Res B Appl Biomater. 2018 Aug;106(6):2295-2306; and Morad et al., 2019. Biomed Mater. 2019 Apr 29;14(4):045005.

Then, the cleaned pieces were ground using either a mortar and pestle or an electrical grinding machine. Both procedures yielded a mixture of grains ranging between 20 μm and 200 μm in size. The grains were then passed through an electrical sieving strainer using a filter mesh of 40 μm. Particles having a size of 40 microns or less were used.

The obtained coral skeleton grains (25 milligrams) were incubated overnight at 4° C. with 0.5 ml of a 20 μg/ml PDL in aqueous solution at pH 8.5. The obtained coral skeleton grains having the PDL associated therewith (e.g., at least partially coating the grains), were then washed with water, dried, and incubated for 5 minutes at room temperature with 0.5 mL of a 2.5% sodium citrate aqueous solution (1.25 mg sodium citrate). The obtained grains were then washed and dried in air, in a biological hood. This exemplary composite material is also referred to herein interchangeably as CPC or as Mixed CS (small)-PDL-C.

While the interactions associating the positively charged PDL to the crystalline coral skeleton particles and the citrate are yet to be determined, it is assumed, without being bound by any particular theory, that the negatively charged carbonate is associated with the positively charged amine groups of the lysine side chains and/or the terminus amine, possibly via electrostatic and/or hydrogen bond interactions, and the citrate molecules are bound to the positively charged amine groups of the lysine side chains and/or the terminus amine, and/or to the calcium of the calcium carbonate possibly via electrostatic interactions and/or hydrogen bond interactions, and/or to the C-terminus, by means of hydrogen bond interactions.

Using the above procedure, other composite materials, prepared using calcium carbonate particulate material from other sources and/or featuring other particles average size, and/or PDL in other concentrations and/or featuring other molecular weights, and/or polymeric and non-polymeric associating moieties other than PDL, are prepared.

Exemplary composite materials are generally prepared in accordance with the procedure described hereinabove, while using a citrate salt with any combination of one or more of the calcium carbonate-containing materials presented in Table 1 and one or more of the associating moieties presented in Table 2.

In cases where additional components (e.g., as described in Table 3 above) are included in the composite materials, the one or more components are added either while incubating the calcium carbonate particles with the association moiety, or while incubating with the citrate salt, or thereafter.

Some of the composite materials were mixed in vitro, ex-vivo with blood of 1-2 months old mice at a ratio of 300 mg (CPC)/1 ml blood. Upon contacting the blood, the blood is soaked by the powdered composite material and coagulates, typically within 0.5-4 minutes. The liquid blood was then removed, and the CPC was dried for several hours or overnight in a biological hood. Such composite materials are presented herein by the addition of the letter “B”.

Whenever a lysine or polylysine is indicated, it is meant the lysine (α), unless otherwise indicated (e.g., for lysine-ε).

Tables 4A and 4B below present exemplary composite materials.

TABLE 4A

| | |
|---|---|
| ACC-PDL- | Amorphous calcium carbonate + poly-D-lysine (30-70 kDa, |
| C | 20 μg/ml) + citrate |
| | Amorphous calcium carbonate + poly-D-lysine (30-70 kDa, |
| | 2000 μg/ml) + citrate |
| | Amorphous calcium carbonate + poly-D-lysine (30-70 kDa, |
| | 200 μg/ml) + citrate |
| ACC-L- | Amorphous calcium carbonate + L-lysine + citrate |
| lysine-C | |
| CS-PDL- | Single/Mixed Coral skeleton (small) + poly-D-lysine (30- |
| C | 70 kDa, 200 μg/ml) + citrate |
| CS-PDL- | Single Coral skeleton (large) + poly-D-lysine (30-70 |
| C | kDa, 200 μg/ml) + citrate |
| B-ACC- | Amorphous calcium carbonate + poly-D-lysine (30-70 kDa, |
| PDL-C | 200 μg/ml) + citrate + blood |
| B-CS- | Single Coral skeleton (mix particles) + poly-D-lysine |
| PDL-C | (30-70 kDa, 200 μg/ml) + citrate + blood |

TABLE 4B

| | |
|---|---|
| CPC A | Amorphous calcium carbonate (ACC) + poly-D-lysine (30-70 |
| | kDa) + citrate |
| CPC B | Amorphous calcium carbonate (ACC) + poly-L-lysine (30-70 |
| | kDa) + citrate |
| CPC C | Single Coral skeleton (large or small, as indicated) + poly-D- |
| | lysine (30-70 kDa) + citrate |
| CPC D | Amorphous calcium carbonate + L-lysine + citrate |
| CPC E | Amorphous calcium carbonate (ACC) + poly-L-lysine (30-70 |
| | kDa) + citrate |
| CPC F | Amorphous calcium carbonate (ACC) + poly-L-lysine (>300 |
| | kDa) + citrate |
| CPC G | Amorphous calcium carbonate + poly-L-ε-Lysine (4 kDa) + |
| | citrate |
| CPC H | Amorphous calcium carbonate + poly-L-Arginine (15-70 kDa) + |
| | citrate |
| CPC I | Amorphous calcium carbonate + poly-Ornithine (30-60 kDa) + |
| | citrate |

### Example 2

### Characterization and In Vitro Activity Assays with Blood Samples Drawn from Mice In Vitro Coagulation Effect

In preliminary studies, the effect of an exemplary newly designed composite as described in Example 1 on coagulation was tested.

Blood drawn from 1-2 months old mice was added to test tubes containing the following compositions, at 300mg/ml final concentration:

A CPC composite as described herein, containing ACC bound to PDL and citrate (denoted ACC-PDL-C);

ACC bound to PDL (denoted ACC-PDL); and

Mixed coral skeleton particles (mix) bound to PDL and citrate (denoted CS-PDL-C).

The results are presented in FIG. 2A.

In the control tubes, time to coagulation was about 14 minutes (not shown), while for ACC-PDL-C the time was 1.7 minutes, for ACC-PDL 3 minutes and for CS-PDL-C 0.4 minutes.

These results clearly show that using various composite materials according to some of the present embodiments resulted in bleeding arrest in less than 5 minutes and even in less than a minute.

For checking CPC's toxicity level, it was tested whether the reaction it takes in blood is exothermic. CPC was added to blood as described in the previous section, and the blood temperature was measured before the CPC addition and during the following 10 minutes. No statistically significant differences were detected (not shown), indicating the reaction is non-exothermic.

In additional studies, CPC D (50 mg/ml) or ACC (50 mg/ml) were placed in the center of 30 mm plastic petri dishes and then covered with 300 ml of fresh mice blood. The dishes were tilted (at approximately)10° every 30 seconds to check for the level of liquidity. Once the liquid jellified the time was considered as the TBC.

The obtained photographs and data are shown in FIG. 2B (upper and lower, respectively), demonstrating the effect of the CPC on the TBC. It can be seen that ACC substantially attributes to the decrease on coagulation time compared to the control.

### Example 3

### In Vivo Assays in Mice

Head Bleeding in Mice:

The effect of the exemplary CPC described in Example 1 on bleeding brain wound was tested. A 2-mm deep hole was drilled in cortex of 2-3 months old anesthetized mice using a 2.3-mm diameter drill. Although the wound was relatively small, it bled the entire measurement (up to 30 minutes, not shown). However, when a powder of the exemplary CPC was deposited on the wound, it absorbed quickly the excess of blood and stopped the bleeding within 1-2 minutes.

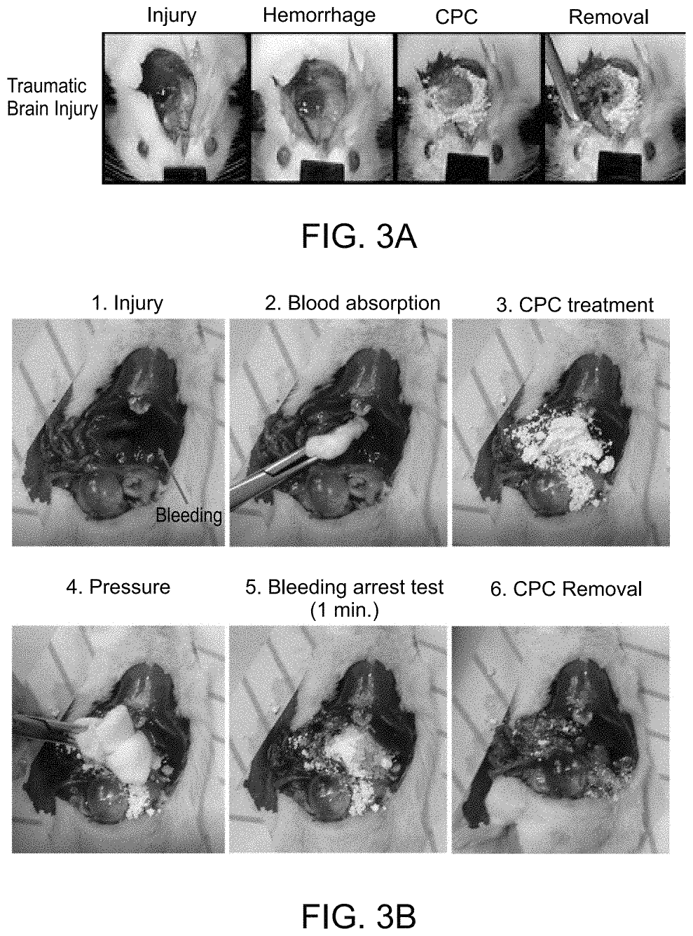

FIG. 3A presents photographs of the brain wound taken 1 minute post-injury after washing excess blood (left); of the bleeding observed 1 minute thereafter (Second to left); after application of a CPC powder (second to the right, taken one minute after its left photo); and after removal of excess CPC (right).

Abdominal Bleeding in Mice:

To check if CPC can arrest a stronger bleeding, abdominal bleeding was tested. Mice at the age of 2-3 months were dissected in the abdomen midline to expose internal organs and photographs were taken during the test. The portal vein was cut, and bleeding was observed, filling very quickly (seconds) the entire abdomen space (see, FIG. 3B, panel 1). The first step in treating the bleeding was rapid absorption of the excess blood using cotton (FIG. 3B, panel 2) followed by immediate pouring of the exemplary CPC powder (FIG. 3B, panel 3) and applying pressure on the injury area, on the powder, using a gauze (FIG. 3B, panel 4). After a minute the pressure was released, and no bleeding was observed (FIG. 3B, panel 5). The excess CPC was then removed with forceps, reveling the abdomen organs and the lack of hemorrhage (FIG. 3B, panel 6).

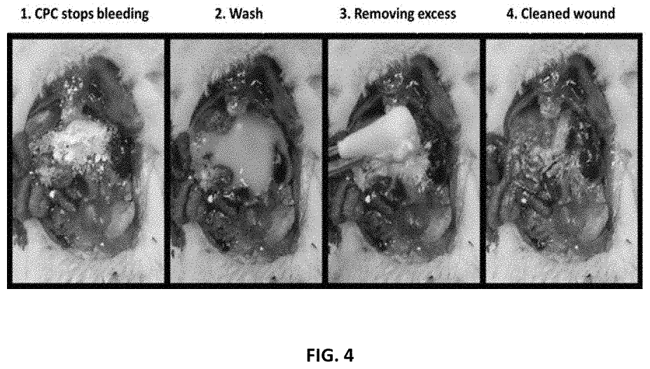

To improve the method of washing out the excess CPC, a different procedure was tested. Instead of using forceps, a quick and gentle wash of the abdomen with saline solution, after bleeding arrest by the CPC, was performed and, as demonstrated in FIG. 4, was shown to be very effective. The excess CPC intermingled with the saline (panel 2) which turned whitish and could be easily soaked out using a gauze (panel 3). This procedure left the abdomen cleaner (panel 4) than the forceps procedure.

### Example 4

### In Vivo Assays in Swine

Herein throughout, the terms “pig” and swine” are used interchangeably.

Arresting Massive Bleeding from Femoral Veins in Pigs:

Two male swines (48-51 kg, Danish Landrace×Large White crossbred swines (Sus domestica) from the domestic herd at Lahav Laboratories, Negev, Israel) were anesthetized and their femur veins exposed (FIG. 5A). A puncture of 2.75 mm diameter in the vein, produced by a canula (FIG. 5B), was followed by a strong and sustained bleeding (approximately 1 ml/sec) (FIG. 5C). After 2-4 seconds of bleeding an Amorphous calcium carbonate-poly-D-lysine-citrate (ACC-PDL-C; CPC A) powder was applied (5-10 grams/ml) blood) (FIG. 5D). A pressure with a gauze was applied for 2-4 minutes, resulting in complete arrest of the bleeding (FIG. 5E). The blockade was stable and preserved the entire experiment—3 hours (FIG. 5F).

The powder appeared partly moisturized with blood (FIG. 5E) and solidified. Yet, it was easily removed using a spatule, and the vein could be re-exposed. When the ACC-PDL-C was removed 10-20 minutes following its application, the vein re-bled. When the CPC was maintained for 3 hours the injury closed (FIG. 5F).

The bleeding arrest produced by the ACC-PDL-C was resistant to body shaking. Bending the femur (FIG. 6A) or the entire leg (FIG. 6B) did not break the ACC-PDL-C blockade (FIG. 6C).

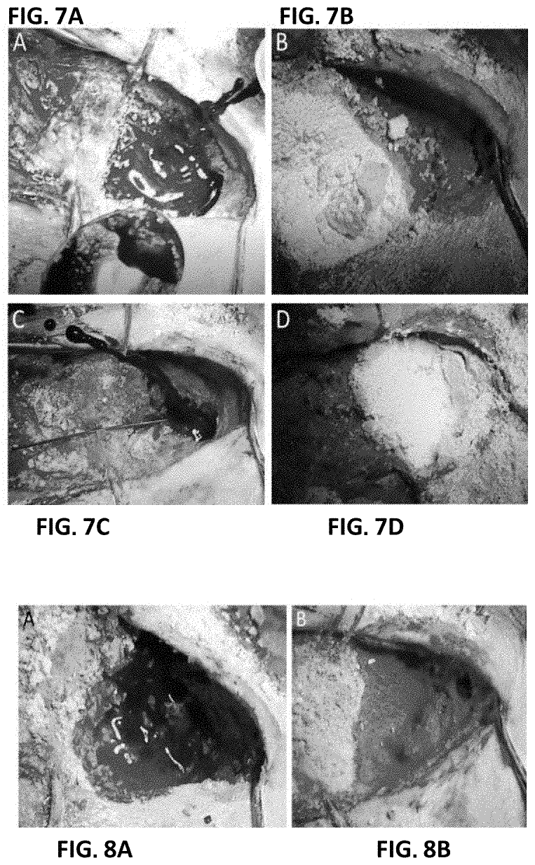

An ACC-PDL-C (CPC A) composite material was used similarly 3 and 4 weeks after its preparation. As shown in FIGS. 7A-D, bleeding was stopped but required application of pressure by gauze for about 4 minutes.

A B-AAC-PDL-C composite material (see, Table 4A) was used similarly. As shown in FIGS. 8A and 8B, the massive bleeding was stopped upon 2 minutes application of pressure.

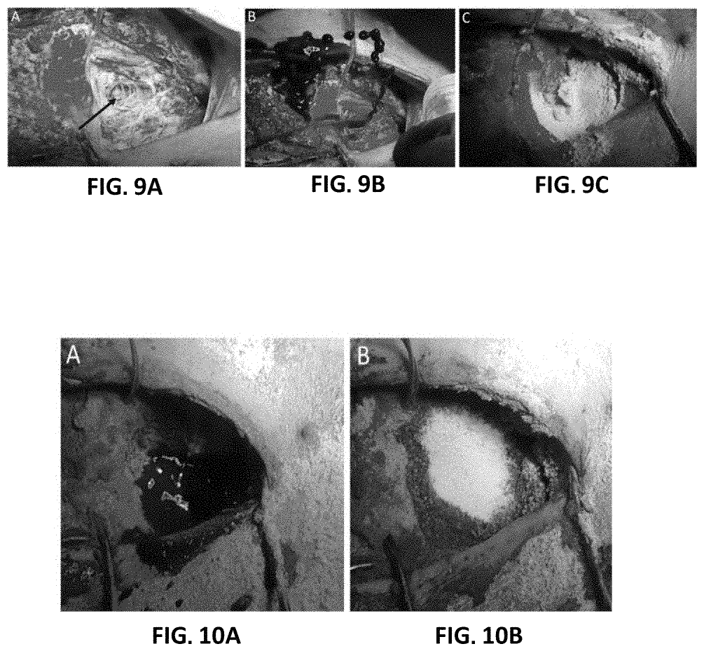

An ACC-L-Lysine-C (CPC D) composite material (see, Table 4A) was used similarly. As shown in FIGS. 9A-C, the massive bleeding stopped upon application of pressure for 2 minutes. The amount of ACC-L-Lysine-C required to stop bleeding was lower by at least two folds from that of ACC-PDL-C.

The same procedure was practiced using a Single CS(mix)-PDL-C composite material (see, Table 4A). As shown in FIGS. 10A and 10B, a complete arrest of massive bleeding from the femoral vein was achieved and required only 1 minute of pressure application. The amount applied was similar to that of the ACC-PDL-C.

Arrest of bleeding from the Jugular veins was also achieved (not shown).

Femoral Artery Massive Bleeding in Swines:

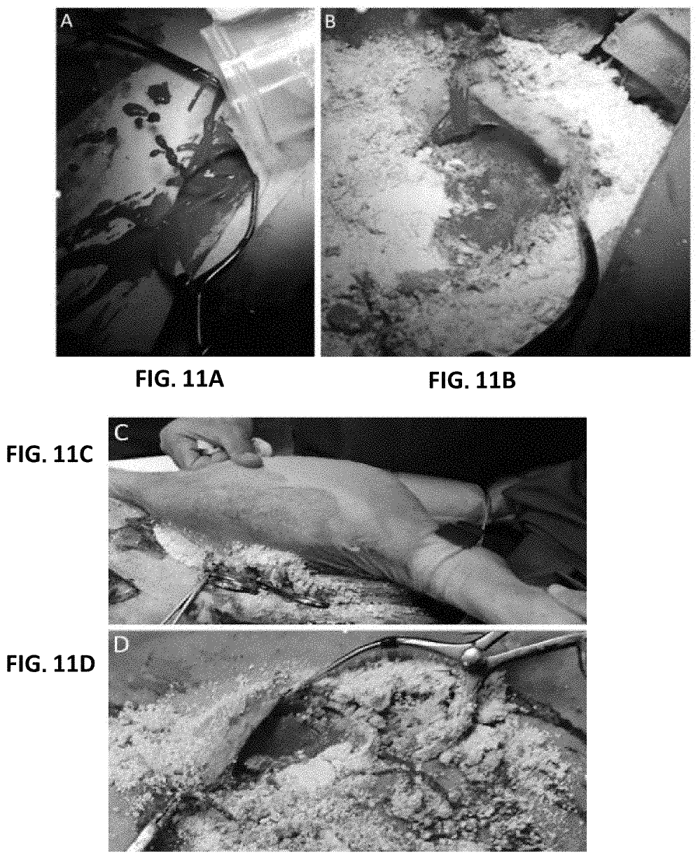

Two male swines (48-51 kg) were anesthetized and their femur arteries were exposed and punctured to produce massive bleeding (between 2.5-5 ml/seconds), as described hereinabove (FIG. 11A). After 2-3 seconds of bleeding 7-10 grams of ACC-PDL-C (CPC A) powder per 1 ml of blood was applied. A pressure with a gauze was applied for 4 minutes, resulting in complete arrest of the bleeding (FIG. 11B). The blockade was stable the entire experiment—3 hours (not shown).

The bleeding blockade was resistant to body shaking, as shown in FIGS. 11C and 11D.

Arrest of bleeding from the Carotid artery was also achieved (not shown).

Arresting Liver Massive and Sustained Bleedings in Pigs:

Livers of Danish Landrace x Large White crossbred pigs (Sus domestica) from the domestic herd at Lahav Laboratories, Negev, Israel were cut (1-3.5 cm length, 0.5-1 cm deep) and the blood was let to flow for 1-30 seconds. Bleeding was sustained, having a rate ranging between 0.1-1 ml/seconds.

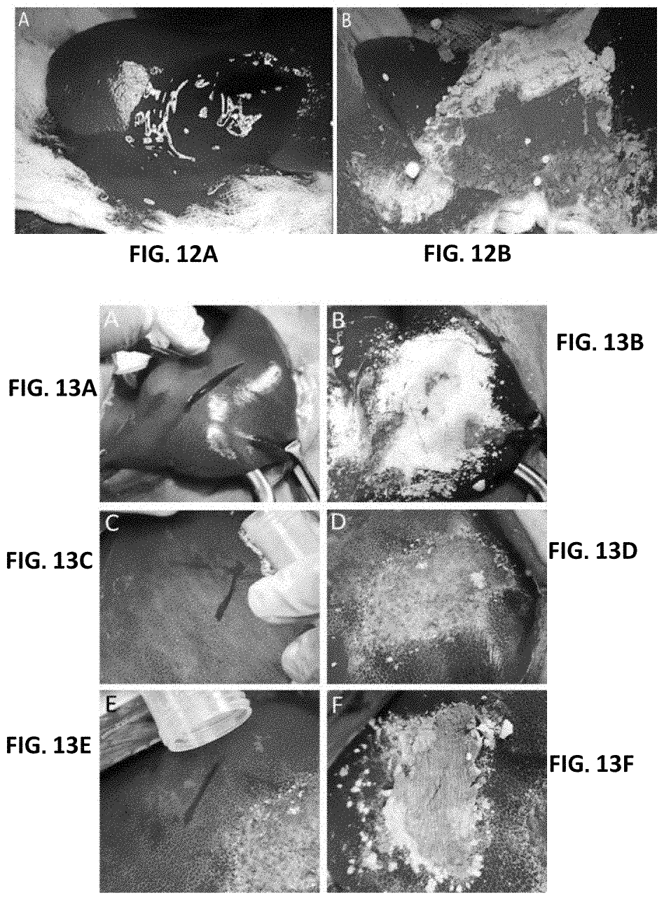

As shown in FIGS. 12A and 12B, massive liver bleeding (FIG. 12A) was stopped upon application of ACC-PDL-C after 2 minutes of pressure application (FIG. 12B).

As shown in FIGS. 13A-B, a continuous bleeding from a 3.5cm (length)×0.5cm (depth) wound in the pig's liver (FIG. 13A) was also stopped by application of ACC-PDL-C (FIG. 13B).

Bleedings from similar wounds were similarly arrested by B-ACC-PDL-C (FIGS. 13C and 13D) and by CS(small)-PDL-C (FIGS. 13E and 13F).

Arresting Sustained Spleen Bleeding in Pigs:

Spleen of Danish Landrace×Large White crossbred pigs (Sus domestica) from the domestic herd at Lahav Laboratories, Negev, Israel were cut (1-3.5 cm length, 0.5 cm deep) and the blood was let to flow for 1-30 seconds. Bleeding was sustained, having a rate of approximately 0.15 ml/second.

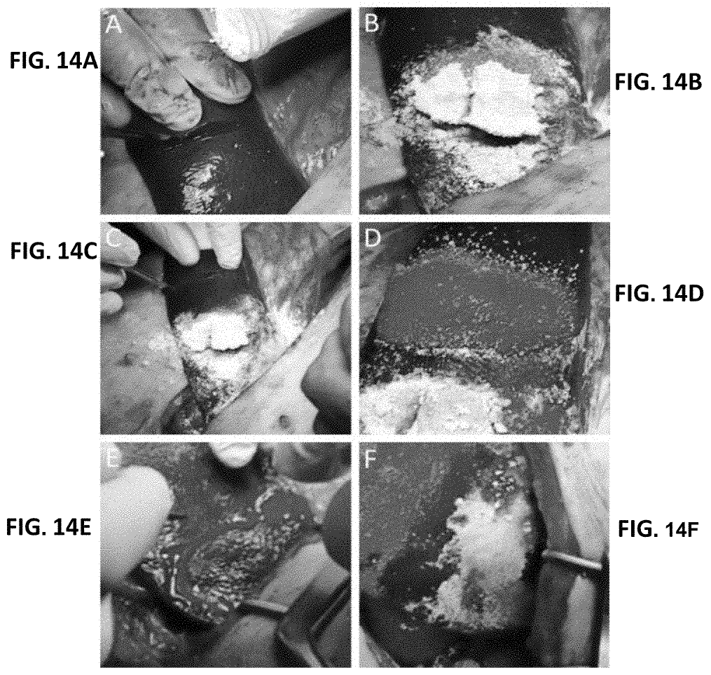

The following powdery composite materials were applied: ACC-PDL-C, CS (large)-PDL-C and CS(small)-PDL-C. The obtained data is shown in FIGS. 14A-F. The first two composite materials stopped sustained bleeding from the spleen upon 1 minute of pressure application (FIG. 14A-D) and the CS (small)-PDL-C stopped this bleeding upon 0.5 minute of pressure application (FIGS. 14E and 14F).

Arresting Superficial Wound Bleeding in Pigs:

Cuts (1-3.5 cm length, 0.5-1 cm deep) were made on the skin of Danish Landrace×Large White crossbred pigs (Sus domestica) from the domestic herd at Lahav Laboratories, Negev, Israel were made, causing bleeding (approximately 1.5 ml/minute). After 3-4 seconds ACC-PDL-C was applied.

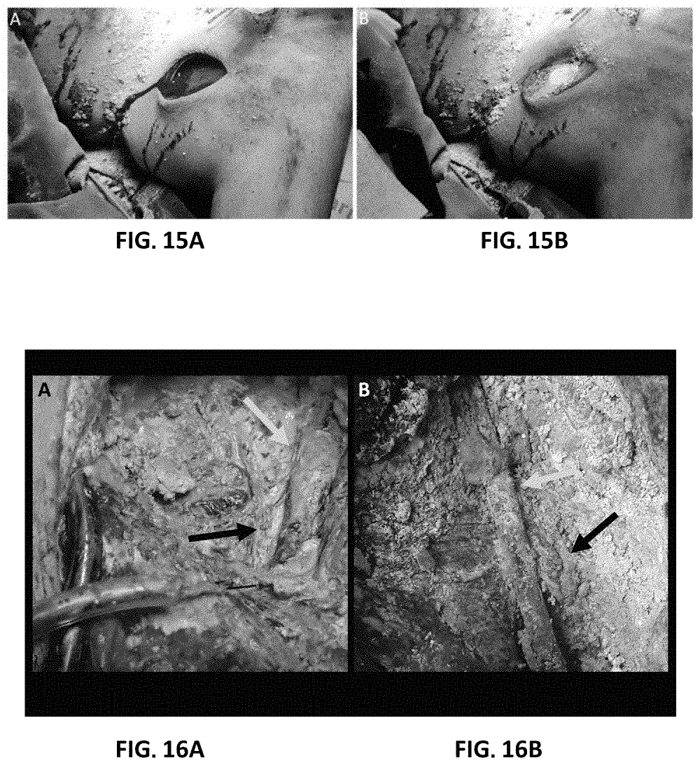

As shown in FIGS. 15A and 15B, ACC-PDL-C blocked bleeding from a superficial wound after 2 minutes of pressure application.

### Example 5

### Comparative Studies

In additional in vivo assays in swines, the effect of various features of the composite material were tested or evaluated.

Evaluating the Role of the CPC Components in Bleeding Arrest:

In order to assess the role of the components of the various CPC composites, various combinations of these components, as individuals or pairs or as a composite were tested in arrest of massive bleeding.

When ACC is combined with Poly-ε-Lysine or L-Lysine, a composite is made as described hereinabove for CPC, without the addition of citrate. When ACC+citrate is used, the composition is as described in WO 2017/046809.

The results, collected from experiments on a femoral vein of a third swine and all done using ACC as the calcium carbonate-containing material, are listed in Table 5 below.

TABLE 5

| | Weight | Time to | | Bleeding | Calculated |
|---|---|---|---|---|---|
| Material | (grams) | arrest | | level | strength |
| ACC | 17.5 | 2.5 | min | 4 | 0.15 |
| ACC + L-Lysine | 7.5 | 47 | sec | 4 | 1.13 |
| ACC + Citrate | 12.5 | 50 | sec | 4 | 0.64 |
| ACC + L-Lysine + Citrate | 7.5 | 33 | sec | 4 | 1.61 |
| ACC + Poly-L-Arginine + | 7.5 | 33 | sec | 3 | 1.21 |
| Citrate | | | | | |
| ACC + L-Arginine + | 7.5 | 40 | sec | 2 | 0.66 |
| Citrate | | | | | |
| ACC + Poly-L-Ornithine + | 7.5 | 33 | sec | 2 | 0.81 |
| Citrate | | | | | |
| ACC + Poly-εLysine + | 7.5 | 30 | sec | 4 | 1.77 |
| Citrate | | | | | |
| ACC + Poly-εLysine | 12.5 | 3.5 | min | 4 | 0.15 |

“Weight”—refers to the amount of materials required to achieve arrest.

“Time to arrest”—the time from application of the material to the end of the applied pressure.

“Bleeding level”—an arbitrary value of the intensity of bleeding (determined visually on a scale of 1 to 5, with 5 being the highest bleeding level and 1 the lowest).

“Calculated strength”—the strength of a material as a bleeding blocker, determined by the following formula:

$$Calculated strength = \frac{Bleeding level}{Weight \times Time to arrest}$$

The results indicate the following:

ACC can arrest bleeding from a swine femoral vein by itself (line 1), however, it is 10.7 times weaker that ACC-L-lysine-C (line 4), and weaker than all other materials tested. “Weaker” means it requires higher amounts and longer pressure time to reach arrest.

Citrate enhances the ability of ACC (rows 3 vs 1) and of ACC-L-lysine (rows 4 vs 2) to arrest bleeding.

ACC-Poly-□□□Lysine-C and ACC-L-lysine-C are strong bleeding blockers.

CPC containing Arginine (row 5) or Ornithine (row 7) can arrest bleeding, but are less active than ACC-L-Lysine-C or ACC-Poly-□□□Lysine-C.

In additional experiments, the effect ACC (A), ACC+citrate (B), ACC+Poly-L-lysine (MW—30-70 kDa) (Polymer 2) (C), and CPC E was evaluated (D).

Swine femoral vein was injured using a 2.75 diameter needle. The tested materials were added (5, 7.5, 12.5 grams each) onto the injury followed by 2 minutes pressure with a gauze.

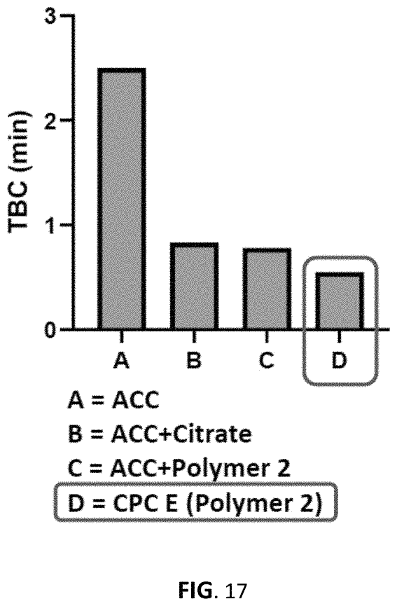

The data obtained for a dose of 12.5 grams for A, B and C and 7.5 grams of D is shown in FIG. 17, and clearly shows the superior performance of CPC E, which exhibits the shortest time to bleeding cessation (TBC) at a lower dose.

Evaluating the Effect of Dosage and Injury Size:

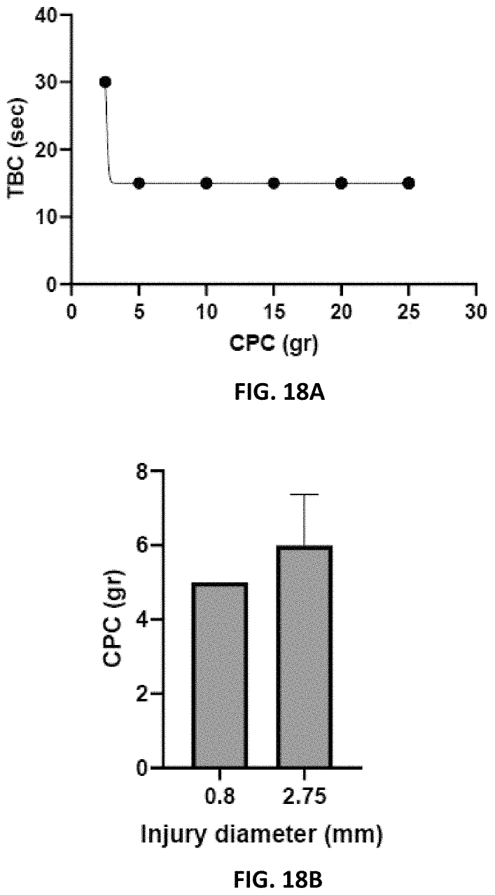

Swine femoral vein was injured using 0.8 or 2.75 diameter needle. Various doses (2.5-5 grams) of CPC D (ACC-L-lysine-citrate) were added onto the injury followed by 2 minutes pressure with a gauze. The obtained data is shown in FIGS. 18A-B.

FIG. 18A shows that for a 0.8-diameter injury, the minimal TBC is already achieved at 5 grams. As shown in FIG. 18B, the minimal effective amount is proportional to the wound diameter.

Evaluating the Effect of the Particles Size:

Swine liver was cut (3.5 cm long, 0.5 cm deep) and CPC or ACC having varying particles size (5 grams) were added onto to the injury, followed by 2 minutes pressure with a gauze.

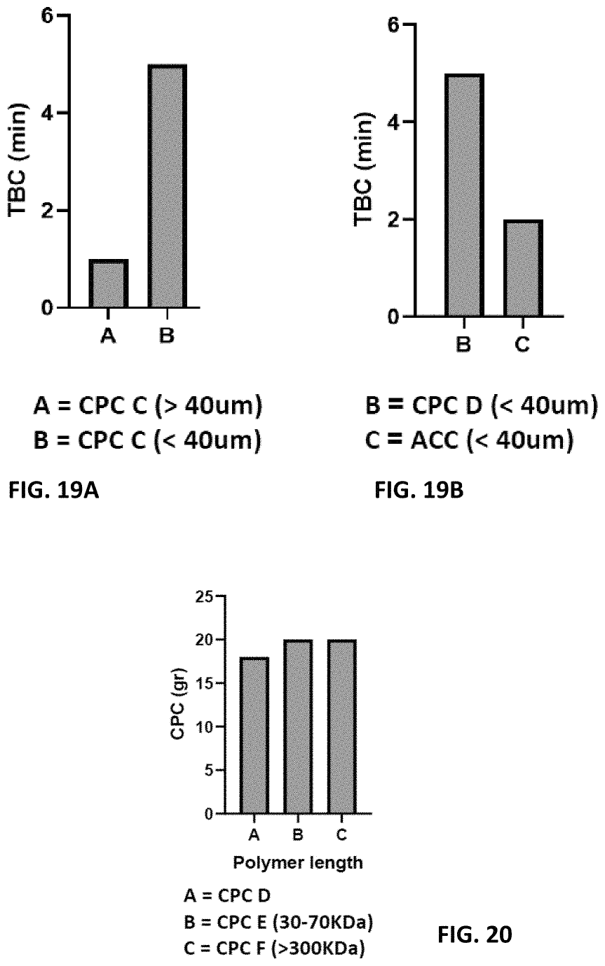

The obtained data is shown in FIGS. 19A-B and show that higher grains size results in shorter TBC, and further show the advantages of including an associating moiety (e.g., a polymeric associating moiety) over using ACC without it.

Evaluating the Effect of Associating Moiety Length (MW):

Swine femoral vein (n=1) was injured using a 2.75 diameter needle. The tested composite material (CPC D (lysine monomer), CPC E (PLL MW 30-70 kDa) and CPC F (PLL MW >300 kDa) was added at elevating doses (5, 12.5, 20 or 25 grams) onto the injury, followed by 2 min pressure with a gauze. The minimal amount required to achieve TBC of 15 minutes post injury was determined for each composite material.

FIG. 20 presents the obtained data and shows a minor advantage for a polymeric associating moiety over a monomeric associating moiety, with no substantial difference as a result of the polymer MW.

Evaluating the Effect of Associating Moiety Type:

Swine femoral vein was injured using a 2.75 diameter needle. Various composite materials having different polymeric associating moieties were added onto the injury (5 grams; n=1), followed by 2 minutes pressure with a gauze.

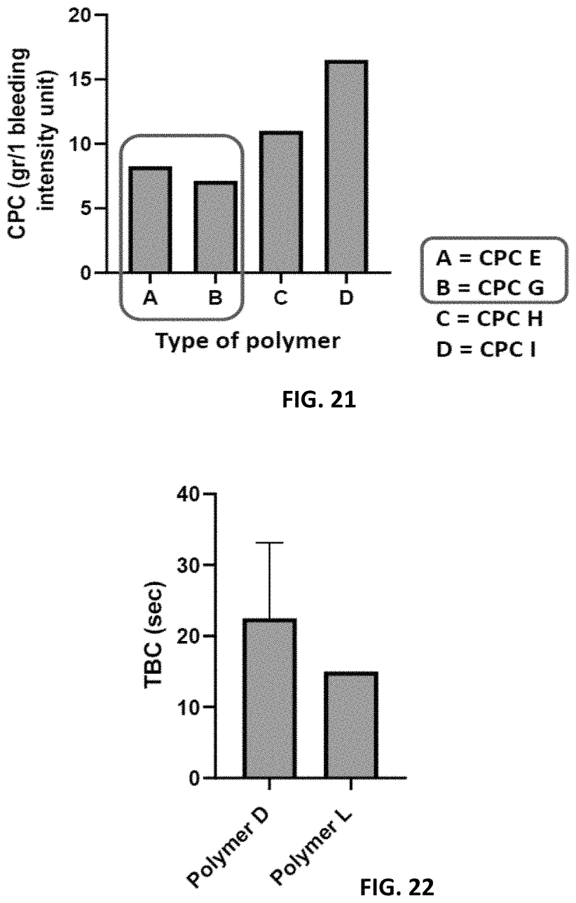

The obtained data is shown in FIG. 21, as the TBC normalized to bleeding severity, demonstrating an improved performance of polylysine associating moiety.

In another assay, swine femoral vein was injured using a 2.75 diameter needle. Various composite materials having polylysine associating moieties differing in configuration (PDL and PLL) were added (20 grams) onto the injury (n=2), followed by 2 minutes pressure with a gauze.

The obtained data is shown in FIG. 22, as the TBC following a dose of 20 grams, demonstrating an improved performance of poly-L-lysine associating moiety.

### Example 6

### In Vivo Studies in Swines Using Composite Material-Containing Bandage Preparation of CPC-Containing Bandage

A gauze (7.5×7.5 cm) was unfolded and laid perpendicularly on a second unfolded gauze (same size) and 5 grams of CPC E (see, Table 4B) were spread on it. Two additional unfolded gauzes were laid on the powder and a second layer of 7.5 grams CPC was added, followed by addition of 5 folded gauzes. The edges of the gauzes were folded and tied to form a closed bandage.

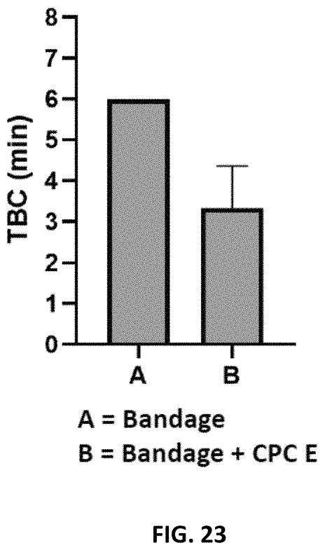

Swine femoral vein was injured using a 2.75 diameter needle. Control and CPC bandages were added onto the injury followed by 2 minutes pressure. The obtained data is shown in FIG. 23, showing the improved performance (shorter TBC) of the CPC-containing bandage.

### Example 7

### Safety

CPCs Toxicity Evaluation:

Table 6 below presents the values measured for various physiological parameters monitored at the start and the end of the experiment (a period of 4 hours) in three pigs (Danish Landrace×Large White crossbred pigs (Sus domestica) from the domestic herd at Lahav Laboratories, Negev, Israel). As shown therein, all parameters retained normal variations.

TABLE 6

| Parameter | Start of experiment | End of experiment | Units |
|---|---|---|---|
| pH | 7.483 | 7.460 | |
| PCO₂ | 37.0 | 43.9 | mmHg |
| PO₂ | 312 | 353 | mmHg |
| BEecf | 4 | 7 | mmol/L |
| HCO₃ | 27.7 | 31.2 | mmol/L |
| TCO₂ | 29 | 33 | mmol/L |
| sO₂ | 100% | 100% | |
| Na | 141 | 137 | mmol/L |
| K | 3.5 | 4.6 | mmol/L |
| iCa | 1.35 | 1.36 | mmol/L |
| Glu | 100 | 137 | mg/dL |
| Hct | 26 | 25 | % PCV |
| Hb (Via Hct) | 8.8 | 8.5 | g/dL |
| HR | 116 | 85 | #/min |
| SₚO₂ | 99 | 100 | % |
| Temp | 36.9 | 38.0 | ° C. |
| Iso | 2 | 1.7 | % |
| BP | 87/71 | 65/42 | mmHg |
| Et CO₂ | 48 | 41 | |

No toxic effect of CPC on various blood components.

Safety Experiment (Swine):

Two swines were anesthetized, their femoral vein was exposed, and a cut made to the vessels using 2.75 mm diameter cannula, causing massive bleeding. ACC-L-Lysine-citrate composite (CLC) was applied in three consecutive sessions at the following amounts—2.5, 5, and 5 grams, with a 15 second inter-session intervals while applying pressure on the wound using a gauze. Note that bleeding cessation took place following applying pressure on the wound using a gauze. Following the last CLC application a pressure was applied with a gauze for 5 minutes. The bleeding arrest was verified by observing for any color and shape changes of the powder. This check was performed every 15 minutes, 5 times. Then the wound was sutured, and the animals were returned to their cages for 2 days.

Blood samples were collected prior to, during and following the surgery (day 1, day 2) and tested for: Blood counts chemistry (liver functions, kidney, CPK) and clotting (PT/PTT, Dfunctions, kidney, CPK) and clotting (PT/PTT, Dfunctions, kidney, CPK) and clotting (PT/PTT, D-dimer). The physical and behavioral condition of the animals was monitored every day, including respiratory system, gastrointestinal system, renal function, eating and drinking, body weight, heart rate, body temperature blood pressure. At the end of the experiment the suture was opened, injury photographed and tissue samples for the injury site, the control (non-injured femoral vein, liver, spleen, lungs, kidneys and heart were injured) femoral vein, liver, spleen, lungs, kidneys and heart were extracted and soaked PFA/formalin, for histology measurements. The PFA/formalin blocked samples were then embedded in paraffin, sectioned and stained for Hematoxylin/Eosin, monitored on a light microscope and photographed. More specifically, following euthanasia, the punctured (treated vein) and contralateral vein were harvested, as well as additional samples from end-organs, and fixed in formalin 10%. Each vein was cut transversely to 2-3 transverse slices. The tissues were processed, embedded in paraffin and sectioned from H&E staining.

The assay is described in FIG. 24A.

All the tested parameters were found to be within the normal range following the treatment (data not shown).

Histopathological analysis found no evidence of thrombosis in filtering organs (kidneys, heart, liver, lungs, spleen), nor was there any evidence of powder in any of the sections of internal organs examined.

FIGS. 24B and 24C present photographs of the treated veins. As can be seen, the powder deposited around the vessel in the area of the adventitia. Fibrin in the intima indicated activation of the coagulation cascade (thrombus formation). Neutrophil infiltration was negligible (FIG. 24B) or moderate (FIG. 24C), apparently associated to the powder material.

### Example 8

### Effect of Treatment Following Traumatic Brain Injury

An open field test was used to assess the levels of animal locomotion in mice. Briefly, 2-3 months old male ICR mice were anesthetized with a retroperitoneal injection of Ketamine:Xylasine (3:1, respectively). Mice were then immobilized to a stereotactic device and an incision was made to expose the area between the Bregma and the Lambda. Craniotomy was performed using a microdrill (RWD) of 2.3 mm diameter cup and the brain will be drilled with a cup of 1.8 mm diameter to induce pTBI. CPC-C implant was implanted into the wound cavity after induction of Q-pTBI, after bleeding subsides using gelatine tampons sponges CPC-C (large particles) was implanted into the brain cavity. Next, the wound was covered with neuro—patch a Suturable Dura Substitution. Then, the scalp was sutured with C17 circle suture needle and 5-0 silk suture.

FIGS. 25A-B present photographs obtained using a Zeiss Primo Vert microscope equipped with the objectives: Plan-Achromat X10/0.25, LD Plan-Achoromat X20/0.3 and LD Plan-Achromat X40/0.5.following TBI (FIG. 25A) and following CPC implantation (FI G. 25B), showing the brain before and after implantation.

Mice were transferred individually into a square arena (50×50×35 cm) illuminated by a dim yellow light. The locomotion of the mice was tracked for 5 minutes by a computerized video system, during which the following behavioral patterns were monitored: time spent at the center of the arena and at the margins, number of rearing and grooming like behavior, total walking distance and averaged velocity. Data collected by EthoVision 10 (Noldus).

The obtained data is shown in FIGS. 25C-D and show that CPC implantation restores memory and locomotion after TBI.

Effect of CPC Treatment on Tissue Regeneration and Synaptic Connectivity Following TBI in Mice:

As described above mice were anesthetized and Q-pTBI was induced. At the end of the experiment, mice were anesthetized and their heart was exposed using a transverse incision and cutting the diaphragm and rib cage. Next, to clean the blood system the superior vena cava was cut and 20 ml of PBSX1 was injected through the left vertical using a 23G needle. Then 20 ml of 4% PFA solution was injected to fixate the animal's organs. After fixation the brains were removed and incubated overnight at 4° C. in 4% PFA solution. Next, the brains were transferred to sucrose solution containing: 58.75% DDW, 30% sucrose, 1.25% PFA and 10% PBSX10 and incubated overnight at 4° C. The brains were then washed with PBSX1, transferred into cryotubes and frozen in liquid nitrogen. To form slices approximately 1.5 mm thick, lateral incisions at both sides of the injury were done while the brain remains frozen. Brain tissue sections (20 μm to 40 μm) of the injured area were prepared using MEV Slee Semi-Automatic Cryostat and collected into 24 well plates filled with PBSx1 with 0.01 Sodium azide.

Then samples were stained as following: Permeabilized with 0.25% TritonX100 for 5 minutes, and blocked with 3% Inactivated Normal Goat Serum (iNGS) for 1 hour. The samples were then incubated overnight at 4° C. with unconjugated primary antibodies. Next, the samples were treated at RT for 1 hour with secondary antibodies. For staining of the nuclei the chemical molecule 4′,6-Diamidine-2′-phenylindole dihydrochloride (DAPI) was added at RT for 15 minutes, then washed three times with Phosphate Buffered Saline X1 (PBSX1). Fluoromount was used as a mounting solution to which 2.5% of 1,4-diazabicyclo[2.2.2]octane (DABCO) was added to improve the lifetime of the samples. Fluorescent images were acquired using an inverted Zeiss Axio-observer Z1 microscope equipped with four objectives: X10/0.13, X20/0.45, X40/0.10, X60/0.85 and with fluorescent DAPI, FITC and Rohdamine filter cubes.

FIG. 26A presents the obtained SV2 images (upper panel) and color coded images of selected magnified areas from the SV2 images (lower panel), showing that CPC A treatment on increases tissue regeneration and synaptic connectivity.

FIG. 26B is a bar graph showing the mean protein expression of SV2. This results further demonstrate the enhanced tissue regeneration and perhaps also synaptogenesis induced by CPC A implantation.

### Example 9

### Platelet Aggregation

Platelet Aggregation Test:

Glass coverslips (round, 12 mm diameter) were covered by grains of CPC D, heat to 80° C. for 10 minutes to cause adherence of the grains. The coverslips were then transferred into plastic petri dishes (30 mm) and loaded with 100 ml fresh mice blood. After 3 minutes incubation at room temperature the dishes were tilted and the liquidous blood removed. After a doubled wash with phosphate buffered saline, the coverslips were soaked in paraformaldehyde (4%) for 10 minutes for fixation. Part of the samples were labeled by immunofluorescence using an antibody to the protein CD41 which is specific for platelets. Other samples were stained by Giemza.

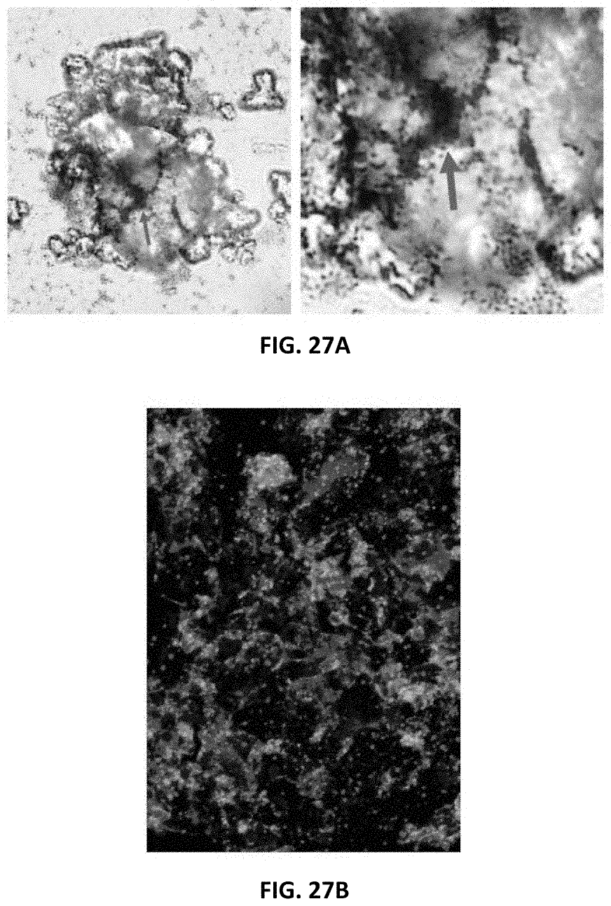

The obtained data is presented in FIGS. 27A-B.

Platelet Aggregation with Collagen-Containing CPC:

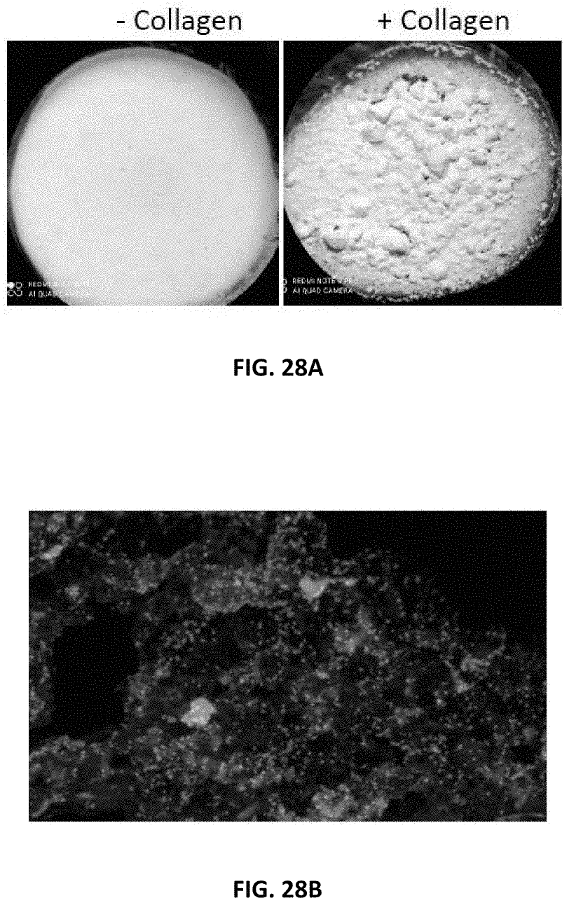

30 grams of CPC D were added to 50 ml collagen solution 0.85 mg/ml (in DDW) and incubated overnight at 4° C. while stirring. Then the solution was centrifuged at 1000 g for 3 minutes and the supernatant was removed. The pellet was washed with 25 ml of DDW per tube, centrifuged again at 1000 g for 3 minutes and the supernatant was removed. The pellet was then dried in a biological hood under ventilation for 24 hours and stored at 4° C. until usage. The same procedure was performed without collagen. Photographs of the obtained pellets are shown in FIGS. 28A-B.

### Example 10

### Concluding Remarks

The data obtained in these studies demonstrate the high efficacy of various exemplary composite materials in arresting both bursting bleeding (veins, arteries) and slower and sustain hemorrhages (liver, spleen), in seconds to minutes.

Some further preliminary insights gained in these studies show that applying the powdery composite material should preferably be made in an amount that covers the entire bleeding area (e.g., 5-40 grams for veins, 60-70 grams for arteries, 10-20 grams for liver and spleen), preferably along with efficient pressure application with a gauze. The amount to be applied can be determined considering the bleeding rate and the amount of blood that leaked prior to treatment. Any type of bleeding, including those with very high pressure, can be arrested, while using the appropriate amount of the composite material and sufficient pressure duration.

Once the powdery composite material is applied onto a bleeding organ or tissue or wound, preferably pressure is applied (e.g., by means of a force applied by a human being), for example, by means of a gauze that covers the wound and optionally its vicinity and is pressurized. The duration of the pressure can vary between seconds to few minutes, depending on the bleeding rate and the size of the bleeding opening. The actual arrest of the bleeding typically takes place 10-30 seconds for veins, liver and spleen, and 50-120 seconds in arteries, upon contacting the bleeding area with the composite material of some of the present embodiments.

Composite materials made of coral skeleton as the calcium source were more absorbent than the ACC-based composite materials and typically stopped the bleeding in half time pressure. The CS large particles were more absorbent that the small particles. CS (small)-PDL-C was shown to be the most efficient powder, succeeding in reduction of vessels bleeding in 1 minute pressure application and liver and spleen injuries in 0.5 minute pressure application.

The “B” (blood soaked) series was generally less efficient but produced a tighter closure of wounds in the liver and the spleen than the ACC-based composite materials.

The obtained data further show that ACC-PDL-C was strong enough to block vessel bleedings and even to close the wound at 3 hours after application.

Testing its shelf life showed that a one-month old powder exhibits reduced blocking capacity, as measured by the pressure time required to complete stop. This powder was also effective in blocking liver and spleen injuries and covered the wound with a solid structure.

ACC-L-Lysine-C was shown to exhibit a higher activity compared with ACC-PDL-C, requiring about 50% less amount and time to achieve complete block. ACC-poly-□-lysine-C showed similar strength to CPC containing L-lysine. As the poly-ε-lysine is known as having anti-bacterial activity, CPC with poly-□-lysine can be used to arrest bleeding and as antibiotic, simultaneously.

The results also indicate that although ACC alone and ACC with L, D or □-Lysine can arrest bleeding, the combination of all three components, the ACC, the citrate and the mono/poly lysine provides an enhanced bleeding arrest effect. See, Table 5 and FIGS. 2A-B.

It was also found that once a powdery CPC (ACC-L-Lysine-C) composite contacts blood in femoral vein injury it becomes pasty, and features a texture of an ointment (FIG. 16A, yellow arrow=vein; black arrow=ointment-like texture). Such a texture may be advantageous in covering blood ruptures, especially at the operating table. Using the compound ACC-L-Lysine, without citrate, the composite remains in a dry, powdery form (FIG. 16B, yellow arrow=vein; black arrow=dry powder texture). It was further found that the change in texture is oppositely correlated with the size of the polymer used (not shown).

Application of CPC composite materials was also shown to arrest bleeding in brain injury to increase regeneration of brain functions following TBI.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composite material comprising a citrate, a calcium carbonate-containing material, and an associating moiety being associated with said citrate and said calcium carbonate-containing material.

2. The composite material of claim 1, wherein said associating moiety is a positively-charged moiety at physiological pH.

3. The composite material of claim 1, wherein said association moiety is a polymeric moiety.

4. The composite material of claim 1, wherein said association moiety is a biocompatible moiety.

5. The composite material of claim 1, wherein said association moiety is a polypeptide.

6. The composite material of claim 5, wherein said polypeptide comprises at least one amino acid residue that is positively charged at physiological pH.

7. The composite material of claim 5, wherein said polypeptide essentially consists of amino acid residues that are positively charged at physiological pH.

8. The composite material of claim 5, wherein said polypeptide is or comprises a polylysine.

9. The composite material of claim 8, wherein said polylysine is selected poly-D-lysine, poly-L-lysine and poly-ε-lysine.

10. The composite material of claim 5, wherein said polypeptide is or comprises collagen.

11. The composite material of claim 1, wherein said association moiety is or comprises lysine.

12. The composite material of claim 11, wherein said lysine is selected from L-lysine, D-lysine and ε-Lysine.

13. The composite material of claim 1, wherein the calcium carbonate-containing material comprises amorphous calcium carbonate (ACC).

14. The composite material of claim 1, wherein said calcium carbonate-containing material is a particulate material.

15. The composite material of claim 14, wherein said particulate material comprises particles having an average particle diameter in the range of from 0.1 micron to 10 millimeter, or from 0.1 micron to 1 millimeter, or from 0.1 micron to 500 microns, or from 0.5 microns to 500 microns, or from 1 micron to 500 microns, or from 5.0 microns to 500 microns.

16. The composite material of claim 1, wherein a weight ratio of said citrate and said calcium carbonate-containing material ranges from 10:1 to 1:10, or from 5:1 to 1:5.

17. The composite material of claim 1, wherein a weight ratio of said association moiety and said calcium carbonate-containing material ranges from 5000:1 to 250:1.

18. The composite material of claim 1, further comprising a swelling polymeric moiety and/or a coagulating agent.

19. A pharmaceutical composition comprising, or consisting of, the composite material of claim 1.

20. The composition of claim 19, being formulated as a topical dosage form.

21. An article-of-manufacturing comprising the composite material of claim 1, the article-of-manufacturing being configured for applying the composite material to a bleeding organ and/or tissue.

22. An article-of-manufacturing comprising the pharmaceutical composition of claim 19, the article-of-manufacturing being configured for applying the composition to a bleeding organ and/or tissue.

23. A method of inducing coagulation of blood, the method comprising contacting blood or a bleeding organ and/or tissue with the composite material of claim 1.

24. The method of claim 23, wherein said contacting is effected in vivo.

25. The method of claim 23, wherein said contacting is with a blood vessel.

26. The method of claim 25, wherein said blood vessel is an internal blood vessel or a blood vessel of an internal tissue.

27. The method of claim 23, wherein said composite material forms a part of a pharmaceutical composition that further comprises a pharmaceutically acceptable carrier or of an article-of-manufacture.

28. A method of reducing or arresting hemorrhaging in a subject in need thereof, the method comprising contacting a hemorrhaging tissue or organ with the composite material of claim 1.

29. A method of treating traumatic brain injury in a subject in need thereof, the method comprising contacting the injury site with the composite material of claim 1.

30. The method of claim 29, wherein said contacting comprises implanting the composite material in the injury site.
